(19) United States
(12) Patent Application Publication
(10) Pub. No.: US 2016/0340743 A1
(43) Pub. Date: Nov. 24, 2016

(54) RECURRENT FUSION GENES IDENTIFIED IN HIGH -GRADE SEROUS OVARIAN CARCINOMA

(71) Applicant: BAYLOR COLLEGE OF MEDICINE, Houston, TX (US)

(72) Inventors: Laising Yen, Pearland, TX (US); Kalpana Kannan, Albany, CA (US)

(21) Appl. No.: 15/117,420

(22) PCT Filed: Feb. 10, 2015
(86) PCT No.: PCT/US15/15284

§ 371 Date: Aug. 8, 2016

Related U.S. Application Data
(60) Provisional application No. 61/938,075, filed on Feb. 10, 2014.

(51) Int. Cl. C12Q 1/68; G01N 33/574; C12N 9/12; C07K 14/705; C07K 14/47

(52) U.S. Cl. CPC C12Q 1/6886; C07K 14/70596; C07K 14/47; C07K 14/4738; C12N 9/12; G01N 33/57449; C12Y 207/11001; C12Q 2600/158; C12Q 2600/106; C07K 2319/00

(57) ABSTRACT

Embodiments of the disclosure include methods and compositions associated with CDKN2D-WDFY2 chimeric RNA, the fusion gene that produces the chimeric RNA, and polypeptides produced from the chimeric RNA. In particular embodiments, the chimeric RNA is useful for methods of treatment, diagnosis, and/or prognosis as they relate to ovarian cancer, or therapy therefor, including at least high-grade serous carcinoma.

This application claims priority to U.S. Provisional Patent Application 61/938,075, filed Feb. 10, 2014, which is incorporated by reference herein in its entirety.

### TECHNICAL FIELD

Embodiments of the disclosure encompass at least the fields of cell biology, molecular biology, medicine, diagnostics, and prognostics.

### BACKGROUND OF THE INVENTION

Ovarian cancer is the most lethal gynecologic malignancy in women. Approximately 225,500 women are diagnosed with ovarian cancer with an estimated 140,200 associated deaths annually (Jemal, et al., 2011). Almost 70% of the ovarian cancer cases are the high-grade serous carcinoma (HG-SC) subtype (Jemal, et al., 2011), which is typically detected at advanced stages due to lack of effective screening tools. HG-SC differs substantially from other subtypes of ovarian carcinoma in their molecular features. Common cancer genes such as TP53 and BRCA1/2 are mutated in 96% and 22% of HG-SC patients, respectively (Bell, et al., 2011). These mutations could contribute to the extensive genome rearrangements and high levels of heterogeneity observed in HG-SC (Bell, et al., 2011). The high degree of heterogeneity in HG-SC suggests diverse clonal lineages within the same patient and among different patients. Discovery of specific molecular signatures for major clonal lineages is essential for understanding the underlying pathogenesis of HG-SC and for designing personalized treatment.

The characteristic massive genome rearrangement in HG-SC implies that recombination events such as gene fusions should be common. If a fusion gene leads to oncogenic consequences, then it will be present in clonal expansions, and therefore, likely recurrent among tumors. Highly frequent gene fusions are significant for several reasons. For example, the BCR-ABL fusion gene in chronic myeloid leukemia is known to initiate oncogenesis through the formation and mis-regulation of a fusion protein (Mitelman, et al., 2007). The BCR-ABL fusion is also a clinical biomarker of high diagnostic and prognostic utility. In addition, this fusion protein serves as a therapeutic target for the drug Gleevec. In prostate cancer, the fusion gene TMPRSS2-ERG was found in 50% of patients, and it is used to classify patient groups (Perner, et al., 2006; Tomlins, et al., 2005). Fusion genes of comparable utility and frequency of occurrence are particularly difficult to identify in HG-SC because of the high heterogeneity observed in these tumors. This difficulty is illustrated by a recent study that identified 45 fusion genes in ovarian cancer, none of which occurred in more than one patient (McPherson, et al., 2011). Another study used transcriptome sequencing to identify a fusion gene, ESRRA-C11orf20, that occurs between neighboring genes and was shown to be present in 15% of patients with HG-SC (Salzman, et al., 2011). Yet, it is unknown whether this fusion gene translates into a fusion protein or is cancer-specific as its presence/absence in non-cancerous tissues was not reported.

Embodiments of the disclosure include solutions for long-felt needs in the art of cancer diagnosis, prognosis, and therapy.

### BRIEF SUMMARY OF THE INVENTION

Embodiments of the disclosure include methods for identifying certain types of cancer, including certain types of ovarian cancer. Embodiments of the disclosure provide treatment for cancer, including treatment for particular types of ovarian cancer. Aspects of the disclosure provide methods that allow one to tailor particular treatments for an individual based on determination of the presence or absence of a particular fusion gene from a sample from the individual.

In embodiments of the disclosure, CDKN2D-WDFY2 is a cancer-specific fusion gene recurrent in ovarian cancer, including in high-grade serous ovarian carcinoma. In embodiments of the disclosure, MUC1-KRTCAP2 chimeric RNA or an isolated polypeptide therefrom and/or BCAM-AKT2 chimeric RNA or an isolated polypeptide therefrom are associated with ovarian cancer, including in high-grade serous ovarian carcinoma.

Embodiments of the disclosure provide diagnostic methods for particular types of ovarian cancer, such as the presence or absence of a particular fusion gene being diagnostic for the individual. Other embodiments concern methods for discrimination of particular types of ovarian cancer based on the presence or absence of a particular fusion gene. Certain aspects provide prognosis for an individual for particular types of ovarian cancer upon determination of the presence or absence of a particular fusion gene. One is able to provide prognosis for an individual based on determination of the presence or absence of a particular fusion gene in a sample from the individual. Survival outcomes are predicted based on the presence or absence of a particular fusion gene, in particular facets of the disclosure.

In this disclosure, a strategy combining high-throughput paired-end transcriptome sequencing and stringent bioinformatic filtering was utilized to identify novel fusion genes in HG-SC. Importantly, one of the identified fusion genes, CDKN2D-WDFY2, is present among 20% of 60 cancer samples analyzed and absent in non-cancer samples. This fusion gene is also expressed in OV-90, an established HG-SC cell line. A genomic breakpoint was identified in intron 1 of CDKN2D and intron 2 of WDFY2 in HG-SC patient sample, providing direct evidence that this is a fusion gene. Transfection of this fusion transcript leads to the loss of wildtype CDKN2D and wildtype WDFY2 protein expression, and a gain of a short WDFY2 protein isoform that changes the protein levels of PI3K/AKT pathway members, in at least certain embodiments. This is by far the most frequent HG-SC-specific fusion event that has implications in a major signaling pathway that is known to be important for oncogenesis. The CDKN2D-WDFY2 fusion gene represents a molecular signature important for defining a major sub-lineage of HG-SC and provides crucial insight into the underlying mechanism of this deadly disease.

In some embodiments, there is as a composition of matter, an isolated chimeric RNA of Table 1 or an isolated polypeptide produced therefrom or an isolated MUC1-KRTCAP2 chimeric RNA or an isolated polypeptide produced therefrom. In specific embodiments, the chimeric RNA is CDKN2D-WDFY2. In particular embodiments, there is a substrate comprising polynucleotides attached thereto, said polynucleotides defined as one or more isolated chimeric RNAs of the disclosure. In particular cases, all or greater than 50% , 55%, 60%, 65%, 70%, 75%, 80%, 85%, 90%, 95%, 97%, or 99% of the polynucleotides attached to the substrate are one or more isolated chimeric RNAs of the disclosure. In certain cases, there is a substrate comprising polypeptides attached thereto, said polypeptides defined as one or more isolated gene products of the chimeric RNAs of the disclosure. In embodiments wherein the presence of MUC1-KRTCAP2 chimeric RNA or an isolated polypeptide produced therefrom is assayed, a gene product produced from the chimeric RNA may be secreted, and the secreted product may be detected by standard means in the art.

In one embodiment, there is a method of determining a diagnosis, prognosis, risk for, or treatment for ovarian cancer in an individual, comprising the step of assaying a sample from the individual for the presence of a fusion gene that produces a composition of the disclosure, assaying a sample from the individual for the presence of the composition of the disclosure, or assaying a sample from the individual for the presence of a polypeptide produced from the composition of the disclosure. In particular aspects, assaying for the fusion gene utilizes FISH or long-range PCR. In certain aspects, assaying for the polypeptide utilizes antibodies directed to the polypeptide. In specific embodiments, the ovarian cancer is high-grade serous ovarian carcinoma.

In particular cases, when a sample from an individual comprises the a chimeric RNA encompassed by the disclosure or an isolated polypeptide produced therefrom, the individual has high-grade serous ovarian carcinoma or is at risk for having high-grade serous ovarian carcinoma. When the sample from the individual comprises chimeric RNA encompassed by the disclosure or an isolated polypeptide produced therefrom, the individual is provided a suitable treatment for high-grade serous ovarian carcinoma. The suitable treatment may comprise a therapy that targets the particular chimeric RNA, such as therapy that targets the fusion junction of the particular chimeric RNA. The suitable treatment may comprise a therapy that targets the polypeptide produced from the chimeric RNA.

In some cases, when the sample comprises the CDKN2D-WDFY2 chimeric RNA or an isolated polypeptide produced therefrom, a particular therapy for high-grade serous ovarian carcinoma in the individual will be effective. The therapy may target the truncated WDFY2 protein from the CDKN2D-WDFY2 chimeric RNA or the signal pathway that is affected by the truncated WDFY2 protein, such as the Akt pathway. A therapy may target a truncated protein from the chimeric RNA or the signal pathway that is affected by the truncated protein. When the sample comprises the CDKN2D-WDFY2 chimeric RNA or an isolated polypeptide produced therefrom, a particular therapy for high-grade serous ovarian carcinoma in the individual will be effective, in some cases, including therapy that targets the truncated WDFY2 protein from the CDKN2D-WDFY2 chimeric RNA or the signal pathway that is affected by the truncated WDFY2 protein. In some embodiments, when a chimeric RNA or an isolated polypeptide produced therefrom are identified, they are indicative of a particular therapy not being effective.

In particular embodiments of the disclosure, an assaying step comprises polymerase chain reaction, such as one that amplifies all or part of the chimeric RNA, including the junction of the chimeric RNA. In some cases, an assaying step comprises detection of the polypeptide produced from the CDKN2D-WDFY2 chimeric RNA, such as detection of the polypeptide by antibody.

Some embodiments of the methods further comprise the step of performing an additional cancer detection step on a sample from the individual. In particular aspects, a sample from an individual comprises serum, urine, blood, or biopsy. Some embodiments of the methods of the disclosure further comprise the step of obtaining the sample from the individual.

In particular cases, diagnosis, prognosis, risk for, or treatment for any cancer may be determined using one or more chimeric RNAs of Table 1, using a gene that produces one or more chimeric RNAs of Table 1, and/or using a polypeptide produced from one or more chimeric RNAs of Table 1.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1: CDKN2D-WDFY2 is a highly frequent fusion transcript in HG-SC cancer samples and cell line. (A) The results of nested RT-PCR for CDKN2D-WDFY2 in 60 HG-SC samples (denoted by “S”), 10 non-cancerous donor ovary samples (“OV”), and 4 non-cancerous donor fallopian tube (“FT”) samples are shown. NTC refers to “no template control”. S5 is the sample in which the fusion transcript was initially identified and this serves as the positive control. (B) The results of RT-PCR for CDKN2D-WDFY2 in five HG-SC cell lines (CaOV3, OV90, OVCAR8, OVCAR5 and OVCAR3), in addition to two endometrioid type cell lines (TOV112D and MDAH 2774) are shown.

FIG. 2: CDKN2D-WDFY2 fusion transcript results from a genomic rearrangement of chromosome 19 and 13. (A) CDKN2D gene is present on chromosome 19 and contains two exons while WDFY2 is present on chromosome 13 and contains 12 exons. The RNA junction indicates a fusion between exon 1 of CDKN2D and exon 3 of WDFY2. To identify the genomic breakpoint, a forward primer F1 was designed in exon 1 of CDKN2D and several reverse primers were designed in the intron between exon 2 and 3 of WDFY2. (B) Results from long range PCR on genomic DNA from patient S5 are shown using primer F1 paired with different reverse primers. A product is seen when R2 is used as the reverse primer. (C) Schematic of CDKN2D-WDFY2 genomic breakpoint with the junction sequence and trace identified by Sanger sequencing of the product in (B).

FIG. 3: CDKN2D-WDFY2 fusion transcript gives rise to a short WDFY2 protein isoform. (A) Protein domain structure of CDKN2D and WDFY2. CDKN2D consists of five Ankyrin repeats (AR1-5). WDFY2 contains seven WD-repeats (WD1-7) that are involved in protein-protein interactions and a FYVE domain for binding to phosphatidylinositol-3-phosphate on vesicular membranes. Potential translational consequences of CDKN2D-WDFY2 fusion transcript (plasmid 1) include a truncated CDKN2D protein (7 kD) that starts from the original ATG in CDKN2D ORF (orange) and is fused to an out-of frame exon of WDFY2 (purple), and a short WDFY2 protein (36 kD) that is translated in the original frame starting from an internal cryptic ATG in exon 3 of WDFY2. Plasmid 2 which contains only the ORF for the truncated CDKN2D protein is used as a control. (B) Protein assay of untransfected HEK-293T cells (lane 1 and 4), plasmid 1 transfected (lane 2 and 6), and plasmid 2 transfected cells (lane 3 and 5) with the indicated antibodies. Plasmid 1 transfection led to a 36 kDa protein indicating that short WDFY2 protein isoform is translated (lane 2), while the predicted truncated CDKN2D fusion (7 kD) is not selected for translation (lane 6). Truncated CDKN2D fusion protein is detected only when the expression of plasmid 2 was visualized by anti-FLAG (lane 3) or a commercial anti-CDKN2D antibody (lane 5). Endogenous CDKN2D has a size of 19 kD. Bands at 25 kD and above in lanes 4 to 6 are non-specific bands.

FIG. 4: Proteins that are altered consistently in patient tissues and transfected cell lines by short WDFY2. Heatmaps show the results of a set of 17 proteins identified by RPPA analysis from a total of 130 proteins. These proteins are significantly altered when comparing patient tissue S5 (expressing CDKN2D-WDFY2 fusion transcript) to S19 (does not express fusion transcript), as well as OVCAR8 cell lines transfected with short WDFY2 to that transfected with wildtype WDFY2. The antibodies used in this analysis are indicated on the right. Green and red indicate lower and higher expression on a relative scale. The experiment was performed in triplicates. Arrows point to members of PI3K/AKT pathway which are highly represented in this set of 17 proteins (hypergeometric test p-value=0.0205).

FIG. 5: Recurrence of the 5 validated fusion transcripts in HG-SC. Figure shows the results of nested RT-PCR for the indicated fusion transcripts in 28 high-grade serous cancer samples (denoted by “S”), 10 non-cancerous donor ovary samples (“OV”) and 4 non-cancerous donor fallopian tube (“FT”) samples. NTC refers to “no template control”. In the case of TMEM66-MSRB3, several non-cancerous samples displayed PCR bands. However, only OV1 and OV2 (designated by asterisk) contained the true fusion transcript as confirmed by Sanger sequencing of the bands.

FIG. 6: Truncated CDKN2D protein was not observed in patient sample. Truncated CDKN2D protein can be visualized by a commercial anti-CDKN2D antibody and anti-FLAG antibody when plasmid 2 is transfected. However, band expected from truncated CDKN2D protein is not observed in protein extracts from patient S5.

FIG. 7: Commercial antibodies to WDFY2 do not recognize short WDFY2 protein isoform produced from the fusion transcript. Protein assay of untransfected HEK-293T cells and plasmid 1 transfected HEK-293T cells with the indicated antibodies. All three WDFY2 antibodies did not recognize the short WDFY2 protein isoform (as seen in the FLAG western) but recognized a number of non-specific bands leading to the conclusion that these antibodies could not be used on patient tissues.

FIG. 8: Validation of RPPA results. Western blots were performed using the indicated antibodies to confirm the results from RPPA study. Results confirmed that these three proteins are upregulated in the patient sample S5 (containing CDKN2D-WDFY2 fusion gene) as compared to sample S19 (which does not contain the fusion gene). Similarly, these proteins are upregulated in OVCAR8 transfected with short versus wildtype WDFY2. The differences between the tissue samples are more pronounced than the cell line samples, and this is consistent with the quantitative data obtained by RPPA.

FIG. 9: Controls for RPPA experiment. Western blot using FLAG antibody on OVCAR8 cells transfected with either wildtype WDFY2 or short WDFY2. Similar levels of transfected protein are observed in both cases.

FIG. 10: BCAM-AKT2 is a frequent fusion transcript in HGSC cancer samples. (A) Schematic showing the position of 8 paired chimeric reads aligning to both BCAM and AKT2 genes identified in patient S4. (B) The results of nested RT-PCR for BCAM-AKT2 in 60 HGSC patient samples (denoted by “S”), 25 non-cancerous ovary samples (“OV”), and 9 non-cancerous fallopian tube (“FT”) samples. NTC refers to “no cDNA control”. S4 is the sample in which the fusion transcript was initially identified. It serves as the positive control. (C) Top: Sanger sequencing chromatogram of the RT-PCR bands revealed that BCAM-AKT2 RNA junction joins the 3′ end of BCAM exon 13 to the 5′ end of AKT2 exon 5 (as demarcated by the black line). Middle: Protein reading frame at the RNA junction (green: BCAM, blue: AKT2). Bottom: 8 junction-spanning reads indicated by black lines identified from transcriptome sequencing are shown.

FIG. 11: BCAM-AKT2 fusion is the result of genomic rearrangement. (A) Long range PCR using primers on exon 13 of BCAM and exon 5 of AKT2 on genomic DNA resulted in a band of about 3.5 kb in patient S4, but not in other patients lacking the fusion transcript. (B) Schematic of BCAM-AKT2 genomic breakpoint (*) identified by Sanger sequencing of the genomic PCR product from patient S4. Complete Sanger sequencing results are listed herein. (C) Putative mechanism of genomic translocation leading to BCAMAKT2 fusion.

FIG. 12: BCAM-AKT2 gives rise to an in-frame fusion protein in patient tumor. (A) Protein domains of BCAM/Lu, AKT2, and the predicted BCAM-AKT2 fusion protein. As a result of the gene fusion, BCAM loses the Lu specific region but retains all domains including the transmembrane domain. AKT2 loses the PH domain but retains the kinase domains. Antibodies used against AKT2 and BCAM domains are shown. (B) Upper panel: western blot using AKT2 antibody. Lower panel: western blot using BCAM antibody (after immunoprecipitation with AKT2 antibody). In both cases, a band corresponding to the predicted size of fusion protein (˜110 kDa) was observed in patient S4 but absent in patient S27 or in non-cancerous ovary and fallopian tube. (C) Western blot using phospho-AKT2 antibody indicates BCAM-AKT2 is phosphorylated at serine 474 (lane 3). Controls include Jurkat cells treated with LY294002 (an inhibitor of AKT phosphorylation) or with Calyculin A (to preserves AKT in a phosphorylated state). (D) Western blot indicates the cDNA cloned from patient S4 is translated into a full-length in-frame fusion protein in OVCAR8 cells as visualized by FLAG antibody. It is also phosphorylated as indicated by phospho-AKT2 (serine 474) antibody. (E) Immunocytochemistry of OVCAR8 cells transfected with the BCAM-AKT2-FLAG expression construct. The fusion protein was seen mainly on the cell membrane (arrows) as well as on the protruding filopodia (arrowheads) that form focal adhesions with the extracellular matrix. Deconvolution microscopy image was visualized by FLAG antibody. Image was a projection of seven stacks each with 0.35 μm thickness.

FIG. 13: BCAM-AKT2 is constitutively phosphorylated in transfected OVCAR8 cells. (A) Top panel: Western blot using phospho-AKT2 (serine 474) antibody shows that BCAM-AKT2 remains phosphorylated regardless of the presence/absence of IGF-1 (lane 1 vs. 2). In contrast, the endogenous AKT2 remains largely unphosphorylated and responds to IGF-1 treatment swiftly through phosphorylation (lane 1 vs. 2, and 3 vs. 4). Middle panel: No unphosphorylated BCAM-AKT2 was detected. Bottom panel: actin controls for protein loading. (B) The expressed fusion protein was immunoprecipitated by FLAG antibody and incubated with GSK-3 substrate. Western blot using anti phospho-GSK3 antibody showed that immunoprecipitated BCAM-AKT2 efficiently phosphorylated GSK-3.

FIG. 14: Generation of BCAM-AKT2 fusion in cells via chromosomal translocation leads to focus formation. (A) Schematic showing target sites of designed guide RNAs in BCAM intron 13 and AKT2 intron 4. Dashed red line indicates genomic breakpoint identified in patient S4. Nine combinations of guide RNA pairs were transfected into HEK-293T cells. RT-PCR results show that BCAM-AKT2 fusion transcript were detected in varying degrees depending on the combination of guide RNAs used. (B & C) Upper panels: Examples of foci induced after transfection of BCAM-g3/AKT2-g1 in OVCAR8 and HEK-293T cells. Lower panels: Presence of BCAM-AKT2 fusion transcript as revealed by RT-PCR of foci isolated from transfected OVCAR8 and HEK-293T population. (D) Representative Sanger sequencing of RT-PCRbands (example from focus OVCAR8-46) confirmed that it contains the expected BCAM-AKT2 fusion junction.

FIG. 15: BCAM-AKT2 fusion protein is phosphorylated at threonine 309. Western blot using phospho-AKT antibody specific for AKT isoforms phosphorylated at the threonine site on OVCAR8 transfected extract indicates BCAM-AKT2 is phosphorylated at threonine 309.

FIG. 16: Sanger sequencing confirmation of BCAM-AKT2 fusion transcript generated by designed CRISPR/Cas9 system. Chromatogram of the RT-PCR band obtained from HEK-293T cells transfected with BCAM-g3/AKT2-g1 guide RNAs along with Cas9 shows the expected BCAM-AKT2 RNA fusion junction (as demarcated by the black line).

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein. Embodiments discussed in the context of methods and/or compositions of the invention may be employed with respect to any other method or composition described herein. Thus, an embodiment pertaining to one method or composition may be applied to other methods and compositions of the invention as well.

I. General Embodiments

Ovarian cancer is the fifth leading cause of cancer death in women. Almost 70% of ovarian cancer deaths are due to the high-grade serous subtype, which is typically detected only after it has metastasized. Characterization of high-grade serous cancer is further complicated by the significant heterogeneity and genome instability displayed by this cancer. Other than mutations in TP53, which is common to many cancers, highly recurrent recombinant events specific to this cancer have yet to be identified. High-grade serous carcinoma is the most common subtype of ovarian cancer observed in women, and few ovarian cancer-specific molecular alterations are currently available for targeting and screening. This subtype of ovarian cancer is typically detected at advanced stages (such as after it has metastasized) due to lack of effective early screening tools. Recurrent cancer-specific gene fusions resulting from chromosomal translocations are useful to serve as effective screening tools as well as therapeutic targets.

Using high-throughput transcriptome sequencing of seven patient samples combined with experimental validation at DNA, RNA and protein levels, described herein is a cancer-specific and inter-chromosomal fusion gene CDKN2D-WDFY2 that occurs at a frequency of 20% among sixty high-grade serous cancer samples but is absent in non-cancerous ovary and fallopian tube samples. This is the most frequent recombinant event identified so far in high-grade serous cancer implying a major cellular lineage in this highly heterogeneous cancer. In addition, the same fusion transcript was also detected in OV-90, an established high-grade serous type cell line. The genomic breakpoint was identified in intron 1 of CDKN2D and intron 2 of WDFY2 in patient tumor, providing direct evidence that this is a fusion gene. The parental gene, CDKN2D, is a cell-cycle modulator that is also involved in DNA repair, while WDFY2 is known to modulate AKT interactions with its substrates. Transfection of cloned fusion construct led to loss of wildtype CDKN2D and wildtype WDFY2 protein expression, and a gain of a short WDFY2 protein isoform that is presumably under the control of the CDKN2D promoter. The expression of short WDFY2 protein in transfected cells appears to alter the PI3K/AKT pathway that is known to play a role in oncogenesis. CDKN2D-WDFY2 fusion is an important molecular signature for understanding and classifying sub-lineages among heterogenous high-grade serous ovarian carcinomas. In particular embodiments, CDKN2D-WDFY2 represents a major cellular lineage important for detecting and classifying heterogenous ovarian carcinomas.

In general embodiments of the invention, there are provided methods and compositions concerning chimeric RNAs or associated compositions thereof. In specific embodiments, chimeric RNAs are utilized in methods of diagnosis of one or more types of cancer. In some embodiments, reagents that target the chimeric RNAs are utilized in the treatment of cancer that has cancer cells having one or more particular chimeric RNAs.

In some aspects of the invention, one or more compositions that target one or more chimeric RNAs are employed for the treatment of cancer. In particular aspects, the cancer has been determined to have the chimeric RNA, whereas in other aspects the cancer has not been determined to have the chimeric RNA. The composition may be of any kind, so long as it is able to directly or indirectly targets the chimeric RNA. In specific embodiments, the composition that targets the chimeric RNA is a polypeptide, nucleic acid or small molecule, for example. In some embodiments, the composition that targets the chimeric RNA targets the junction site between the two or more respective components of the chimeric RNA. However, in some embodiments the composition that targets the chimeric RNA does not target the junction site and targets one or the other of the chimeric components. In certain embodiments, the composition that targets the chimeric RNA is an antibody that recognizes the chimeric RNA gene product or, in alternative cases, the chimeric RNA itself. In particular aspects of the invention, the antibody immunologically recognizes the junction site of the gene product. In certain embodiments, the composition that targets the chimeric RNAs or fusion genes comprise one or more antibodies that immunologically recognize the fusion proteins or truncated proteins derived from the fusion genes or chimeric RNAs.

II. Nucleic Acid Detection

In certain embodiments of the invention, one or more chimeric RNAs are assayed for in a cancer sample from an individual. In certain embodiments of the invention, one or more fusion genes in an individual's genome are assayed for in a cancer sample from an individual. The assay may or may not identify the chimeric RNA, depending on the nature of the cancer of the individual. In cases wherein the assay identifies the chimeric RNA or fusion gene, the individual is then determined to have ovarian cancer, is then determined to be at risk for having ovarian cancer, and/or will have a certain prognosis or outcome for therapy for ovarian cancer. In specific embodiments, the ovarian cancer is HG-SC. The chimeric RNA(s) or fusion gene(s) may be detected by any suitable means.

A. Hybridization

Hybridization methods may be employed to detect the chimeric RNA, such as using a polynucleotide that is complementary to at least part of the chimeric RNA. In particular aspects, a polynucleotide is complementary to a region of the chimeric RNA that is unique to the chimeric RNA. Such a region may include a junction site of the two components that defines the chimeric RNA, for example. Fluorescence in situ hybridization (FISH) may be employed to detect the presence of fusion genes in an individual's genome.

The use of a polynucleotide between 10 and 100 nucleotides, for example, between 17 and 100 nucleotides in length, for example, or in some aspects of the disclosure greater than 100 nucleotides in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, nucleotide sequences of the disclosure may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

B. Amplification of Nucleic Acids

The chimeric RNA may be amplified as part of an assay to detect the chimeric RNA. Standard Reverse transcription polymerase chain reaction (RT-PCR) or quantitative real time polymerase chain reaction (qPCR) may be employed to detect the chimeric RNA. Long-range polymerase chain reaction on genomic DNA from patients may be employed to detect the presence of fusion genes in a patient's genome.

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to one or more chimeric RNAs are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

C. Detection of Nucleic Acids

Following any amplification, it may be desirable to separate the amplification product from the template and/or the excess primer. In one embodiment, amplification products, or any polynucleotides, are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

D. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

I. Immunological Reagents and Methods

In some embodiments, antibodies are utilized to detect a protein product, such as a fusion protein or truncated protein, from the chimeric RNA or fusion genes, including, for example, a WDFY2 protein isoform, a BCAM-Akt2 fusion protein, and/or TMEM66-MSRB3 fusion protein.

A. Antibodies

In certain aspects of the invention, one or more antibodies may be utilized to detect one or more protein products of chimeric RNAs and fusion genes of the invention. The antibody may detect the peptide junction of the different regions of the fusion protein or it may detect the region alone.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

“Mini-antibodies” or “minibodies” are also contemplated for use with the present invention. Minibodies are sFv polypeptide chains which include oligomerization domains at their C-termini, separated from the sFv by a hinge region (Pack, et al., 1992). The oligomerization domain comprises self-associating .alpha.-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al., 1992; Cumber, et al., 1992.

Antibody-like binding peptidomimetics are also contemplated in the present invention. Liu, et al., 2003 describe “antibody like binding peptidomimetics” (ABiPs), which are peptides that act as pared-down antibodies and have certain advantages of longer serum half-life as well as less cumbersome synthesis methods.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. As used herein, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. A “humanized antibody” is an antibody comprising a humanized light chain and a humanized heavy chain immunoglobulin.

B. Antibody Conjugates

The present invention further provides antibodies against chimeric RNA fusion proteins, polypeptides and peptides that may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shokri et al., 1991; Silverman et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰, ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

In another embodiment of the invention, the antibodies are linked to semiconductor nanocrystals such as those described in U.S. Pat. Nos. 6,048,616; 5,990,479; 5,690,807; 5,505,928; 5,262,357 (all of which are incorporated herein in their entireties); as well as PCT Publication No. 99/26299 (published May 27, 1999). In particular, exemplary materials for use as semiconductor nanocrystals in the biological and chemical assays of the present invention include, but are not limited to those described above, including group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge and Si and ternary and quaternary mixtures thereof. Methods for linking semiconductor nanocrystals to antibodies are described in U.S. Pat. Nos. 6,630,307 and 6,274,323.

C. Immunodetection Methods

In still further embodiments, the present invention concerns immunodetection methods for binding, purifying, removing, quantifying and/or otherwise generally detecting biological components such as chimeric RNA gene products or the chimeric RNAs themselves. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In particular embodiments, a proximity ligation assay (PLA) is employed. Therein, two primary antibodies are raised in different species, with each recognizing one part of the fusion protein. Species-specific secondary antibodies, called PLA probes, each with a unique short DNA strand attached to it, bind to the primary antibodies. When the PLA probes are in close proximity, the DNA strands can interact through a subsequent addition of two other circle-forming DNA. This assay can be used to detect fusion proteins. (See Söderberg, et al., 2006; Jarvius, et al., 2007).

In general, the immunobinding methods include obtaining a sample suspected of containing chimeric RNA fusion protein, polypeptide and/or peptide, and contacting the sample with an antibody in accordance with the present invention under conditions effective to allow the formation of immunocomplexes.

These methods include methods for purifying chimeric RNA fusion proteins, polypeptides and/or peptides from patients' samples. In these instances, the antibody removes the antigenic chimeric RNA fusion protein from a sample. The antibody will preferably be linked to a solid support, such as in the form of a column matrix, and the sample suspected of containing the chimeric RNA fusion protein antigenic component will be applied to the immobilized antibody. The unwanted components will be washed from the column, leaving the antigen immunocomplexed to the immobilized antibody, which chimeric RNA fusion protein antigen is then collected by removing the chimeric RNA fusion protein or the complex from the column.

The immunobinding methods also include methods for detecting and quantifying the amount of a chimeric RNA fusion protein reactive component in a sample and the detection and quantification of any immune complexes formed during the binding process. Here, one would obtain a sample suspected of containing a chimeric RNA fusion protein and contact the sample with an antibody against the chimeric RNA fusion protein, and then detect and quantify the amount of immune complexes formed under the specific conditions.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any chimeric RNA gene product fusion protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various forms of cancer. Here, a biological and/or clinical sample suspected of containing a chimeric RNA gene product fusion protein, polypeptide, peptide mutant is used.

In the clinical diagnosis and/or monitoring of patients with various forms of disease, such as cancer, the detection of chimeric RNA gene products and/or an alteration in the levels of chimeric RNA gene products in comparison to the levels in a corresponding biological sample from a normal subject is indicative of a patient with cancer. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive.

1. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, the antibodies of the invention are immobilized onto a selected surface exhibiting protein affinity, such as a well in a polystyrene microtiter plate. Then, a test composition suspected of containing the chimeric RNA gene product fusion protein antigen, such as a clinical sample, is added to the wells. After binding and/or washing to remove non-specifically bound immune complexes, the bound chimeric RNA gene product fusion protein antigen may be detected. Detection is generally achieved by the addition of another antibody that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the chimeric RNA gene product fusion protein antigen are immobilized onto the well surface and/or then contacted with the antibodies of the invention. After binding and/or washing to remove non-specifically bound immune complexes, the bound antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the chimeric RNA gene product fusion proteins, polypeptides and/or peptides are immobilized, involves the use of antibody competition in the detection. In this ELISA, labeled antibodies against chimeric RNA gene product fusion protein are added to the wells, allowed to bind, and/or detected by means of their label. The amount of chimeric RNA gene product fusion protein antigen in an unknown sample is then determined by mixing the sample with the labeled antibodies against chimeric RNA gene product fusion before and/or during incubation with coated wells. The presence of chimeric RNA gene product fusion protein in the sample acts to reduce the amount of antibody against chimeric RNA gene product fusion protein available for binding to the well and thus reduces the ultimate signal. This is also appropriate for detecting antibodies against chimeric RNA gene product fusion protein in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are well known in the art.

2. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

II. Pharmaceutical Preparations

In some embodiments of the invention, a cancer is treated with an agent that targets a particular chimeric RNA, and in at least some cases the cancer is identified as having one or more particular chimeric RNAs. Pharmaceutical compositions of the present disclosure may comprise an effective amount of one or more chimeric RNA-targeting agents (such as an antibody or small molecule or siRNA-based drugs) dissolved or dispersed in a pharmaceutically acceptable carrier. Pharmaceutical compositions of the present disclosure may comprise an effective amount of one or more agents that target a gene product of a chimeric RNA (such as an antibody or small molecule) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one chimeric RNA-targeting agent or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The agent that targets the chimeric RNA or a gene product thereof may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The agent that targets the chimeric RNA or a gene product thereof may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure concerns the use of a pharmaceutical lipid vehicle compositions that include the agent that targets the chimeric RNA or a gene product thereof, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the agent that targets the chimeric RNA or a gene product thereof may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the chimeric RNA-targeting agentS are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, chimeric RNA-targeting agentS may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound chimeric RNA-targeting agent may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

III. Kits of the Invention

Any of the compositions described herein may be comprised in a kit. In a non-limiting example, a chimeric RNA, one or more polynucleotides that hybridize to a chimeric RNA, a substrate having a chimeric RNA or one or more polynucleotides that hybridize to a chimeric RNA, a substrate having a gene product of a chimeric RNA, an antibody to a chimeric RNA or its gene product, a cancer therapeutic and/or a siRNA that targets a chimeric RNA may be comprised in suitable container means in a kit. In some embodiments, the kit comprises an agent that targets the chimeric RNA or a gene product thereof.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing components in close confinement for commercial sale. Such containers may include injection or blow-molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

The kits of the present invention will also typically include a means for containing the vials in close confinement for commercial sale, such as, e.g., injection and/or blow-molded plastic containers into which the desired vials are retained.

In some embodiments, an apparatus to obtain a sample from an individual is included in the kit. The apparatus may be one that extracts ovary tissue, blood, and the like from the individual. Detection reagents for the chimeric RNA or its gene product may be included.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention

### Example 1

### Identification of Novel Fusion Transcripts in High-Grade Serous Ovarian Cancer

To identify fusion transcripts that are transcribed from fusion genes, the transcriptome of seven cancer samples from patients with HG-SC was sequenced. The cancer samples are primary tumors from patients that did not receive neoadjuvant chemotherapy prior to removal of diseased ovary. Because there is still debate about the cell of origin of HG-SC, as histologically similar cancers have been identified on the surface of the ovary and fallopian tube (Lee, et al., 2007; Pothuri, et al., 2010), transcriptome sequencing was performed using two control pools: one pool of RNA from ovaries of 20 non-cancerous donors and another pool of RNA from the fallopian tubes of 6 non-cancerous donors. The Illumina Genome Analyzer II was used for sequencing these samples and output sequences of paired 75 or 100 nucleotide sequence reads were generated. In all, 9 lanes of Illumina Genome Analyzer yielded approximately 476 million reads that were uniquely mappable to the human genome.

The strategy for identifying fusion transcripts was to search for paired ‘chimeric’ reads with each read mapping to a different gene either in the genome or transcriptome. To minimize the cases of false positives, the following filters were used. First, an event was considered a fusion transcript only if it was supported by at least two paired chimeric reads. To avoid clonal duplicate sequence reads leading to spurious fusion calls, it was required that the starting genomic positions of the corresponding paired reads be at least 5 base pairs apart. Second, two genes in theory can fuse in four different strand combinations; it was required that the chimeric reads predominantly support only one of the four strand combinations of gene fusion. Third, cases of overlapping genes and homologous genes with shared sequences were filtered out. Lastly, the fusion supporting reads were remapped with BLAT (Kent, et al., 2002) and it was ensured that the mappings could not find better or equally scoring mappings. This strategy led to the identification of nearly 356 putative fusion transcripts from the 7 cancer samples. Interestingly, the ESRRA-C11orf20 fusion transcript, which is reported to be present in 15% of HG-SC patients, was absent in the 7 cancer samples sequenced. This could be due to either the small sample size or low expression of this fusion transcript such that it could not be detected in our samples. Importantly, the strategy was able to rediscover 16 previously identified fusion transcripts in ovarian cancer such as LAMC2-NMNAT2 and MAG-CD22 (McPherson, et al., 2011), indicating that the method is effective. The remainder of the identified 340 putative fusion transcripts have not been described before.

Forty-seven candidates out of 356 putative fusion transcripts were selected for experimental validation based on the following criteria: 1) the fusion transcript should be present in at least two human cancer samples or supported by 3 or more chimeric reads, or 2) the fusion transcript includes one or more genes that are listed in the cancer gene catalog compiled by MSKCC that implies functional association with cancer.

To validate the presence of these candidate fusion transcripts, specific primers were designed with each primer targeting one parental gene, therefore specifically amplifying the fusion transcripts but not the parental gene transcripts. RT-PCR was employed to validate the presence of fusion candidates in the patient samples that were used for paired-end sequencing. For 15 candidates, RT-PCR products were obtained indicating presence of the fusions at detectable levels. In most of these cases, a single RT-PCR band was obtained from the targeted fusion transcript, which was then excised and sequenced by Sanger sequencing. This led to the identification of the exact RNA junction of the fusion transcript (Table 1).

TABLE 1
Junction sequences of validated fusion transcripts in HG-SC samples.
RNA junction sequences as determined by RT-PCR and Sanger sequencing are shown
for each fusion transcript. The junction is denoted by an asterisk. For CDKN2D-WDFY2,
the sequence of the genomic breakpoint is also shown by an asterisk.

| Fusion | | RNA junction sequence identified by Sanger sequencing | |
|---|---|---|---|
| transcript | Chromosome | (5′-> 3′) | SEQ ID NO: |
| CDKN2D- | Chr 19 & Chr | GAACCGGACGTCATCCGACGCCCTCAACCGCTTCGGCAAG | 1 |
| WDFY2 | 13 | ACGGCGCTGCAG*CTCCATGTTCATGCATGTCTTTTAACCC | |
| | | GGAAACAAGAAGACTGTCCATAGGTCTAGACAATGGTACA | |
| BCAM- | Chr 19 | TTGCTGTCTTCTACTGCGTGAGACGCAAAGGGGGCCCCTG | 2 |
| AKT2 | | CTGCCGCCAGCGGCGGGAGAAGGGGGCTCCG*GAGGAGT | |
| | | GGATGCGGGCCATCCAGATGGTCGCCAACAGCCTCAAGCA | |
| | | GCGGGCCCCAGGCGAGG | |
| TMEM66- | Chr 8 & Chr | TGCTGACCGCGGGCCCTGCCCTGGGCTGGAACGACCCTG* | 3 |
| MSRB3 | 12 | GTTGGCCTTCATTCCACGATGTGATCAATTCTGAGGCAATC | |
| | | ACATTCACAGATGACTTTTCCTATGGGA | |
| FAM19A3- | Chr 1 & Chr 3 | CCGGGGGAGGAGTGTAAGGTGCTCCCGGACCTGTCGGGA | 4 |
| LPP | | TGGAGCTGCAGCAGTGGACACAAAGTCAAAACCACCAAG*T | |
| | | TACTCCAATAAGTCCCGCCTCTCCTTCCTCCAGTTTGGCTG | |
| | | GACAAGCTAAGTTTTCACAT | |
| RFX2- | Chr 19 | GGGGACCAACACGCAGCAGCCGCTGCCGCCGCCGCGGGA | 5 |
| CCDC94 | | GCCGCTGCCCGAACTCCCGGCCCGAACTCCAG*GGCCCTG | |
| | | TTGGAGGAAGCCAGAAAGCGAAGACTGCTGGAGGACTCCG | |
| | | ACTCAGAGGATGAGGCTG | |
| NR2F6- | Chr 19 | GCACCACCGGAACCAGTGCCAGTACTGCCGTCTCAAGAAG | 6 |
| MAST3 | | TGCTTCCGGGTGGGCATGAGGAAGGAGG*AGATGGTGCCA | |
| | | CTGAGTCACCTCGAAGAAGAACAGCCCCCAGCACCTGAGT | |
| | | CCCCAGAGAGCCGCGCCC | |
| WDFY2- | Chr 13 & Chr | GTGATGGTGGGATTGTCGTCTGGAACATGGACGTGGAGAG | 7 |
| S1PR5 | 19 | GCAGGAG*GACAGAGGTCTCAGCTCACATCACAAGTCACTA | |
| | | CCTCTGCAGAGAGGTCTTTCCTGCCCACCATCACCATCTCT | |
| | | GTCGTTTGTCACTGGAAT | |
| CRTAC1- | Chr 10 | TTTCGGGTCTCAGAGTGCAGTCGGGGCTACGAGCCCAACG | 8 |
| GOLGA7B | | AGGATGGCACAGCCTGCGTGG*GCTGGTGGAGCCCTGTGT | |
| | | TGAAGATAGTGACACCACAAGTTGGGAAGAGCCTTGGTCC | |
| | | CTGAATCACTGAATCACTG | |
| LAMC2- | Chr 1 | CAGAAGGTGAGAACAGAAGCCCAGAAGGTTGATATCAGAG | 9 |
| NMNAT2 | | CCAAGATCGATGGGGTTGCAGTCCTAGACACTCTGACGCA | |
| | | TTAGACGGCGTCGAGCATCTGATGG*ACCCTGCACTTGATG | |
| | | ACCAGCTGCACACCCGATT | |
| MAG- | Chr 19 | CGGCAGGGGACAACCCTCCCGTCCTGCTCAGCAGCGACTT | 10 |
| CD22 | | TCGCATCTTTCGGGCACCAGAGAAGTAGG*AGATGCTGCC | |
| | | AGGGTCCCTGAAGAGGGAAGACACGCGGAAACAGGTAAAA | |
| | | ATCATTTTGCTTTTATTTTG | |
| HSP90B1- | Chr 12 | AACAGAGCAAGACGAAGATGAAGAAATGGATGTGGGAACA | 11 |
| C12orf73 | | GATGAAGAAGAAGAAACAGCAAAG*ATGAGCTGAGCTGGA | |
| | | CAACTAAAAGCACCTGTAGCAGTTCTCCCCAACACAGCACT | |
| | | GAGCACATCTAGTAGC | |
| SLC25A29- | Chr 14 | GCTCAGCAGAGAACAGTATGGGACCCCCTCACCAGGCCTG | 12 |
| BC014138 | | GAACACCTCCAGCCACAAAGAAGCCAAAG*ATTAAACTCCT | |
| | | TACAGAAGTTAACTCTGCAGACTGAGAAGACGTGAACAGAG | |
| | | AGAGTCAAGGGGAGTGAT | |
| RNF19B- | Chr 1 | GTGAACTAAAGCATAGTGCACGCAAGCACTCGTGCTATGG | 13 |
| BC036308 | | CCGGTTCCATAATCAGTTCCTACAACCCACAGGACAG*CCT | |
| | | AGTATCAGAAGGCCAGGCGAGACTGCAACACTGCTCATCA | |
| | | CCCCGCGGCGTGATCCCTG | |
| C3orf78- | Chr 3 | TAAAACGACTCGGTCGAGCATGTTCACCAGGGCCC*AGATT | 14 |
| PBRM1 | | CAACCTCATCTTCCCTAATTCAATGACACTCACCTGCCCA | |
| RHOBTB2- | Chr 8 | GCGAAGGCGCTGCCGCGAAGACGCCTGGCGCTGCAGTCC | 15 |
| PEBP4 | | CAG*GTGCCCATGGGTTGGACAATGAGGCTGTCACAGCAG | |
| | | CACTGTACG | |
| KRT7- | Chr 12 | TCGTGGATACTGACTGGCTGGAATCGAGACGCCACCTACC | 16 |
| KRT86 | | GCAAGCTGCTGGAGGGCGAGGAGAGCCG*AGTCTGCAAAC | |
| | | TACGGCCACATGCAGGCTTCCTGTTTGTCAATCAAGTTTTC | |
| | | TTGGAACCCAGTCATACCG | |

CDKN2D-WDFY2 Genomic Breakpoint Sequence:

(SEQ ID NO: 17)

ttagcatcttctctgggaggattcctggttctctgagtgccggggattc
actttccctggggaggttcttattccgtgggtgcgggttcagctccttt
ggatgccgtttccatggggacggctcacgtttcttggggcttctatggg
gagatccgatctctggaaggagaggtcctgtctctaggggttcctcatt
ttgtagggggctctctgagacaaatctggttacttgggggtggggtctt
cattcctaaaggttatgcggatccttgatatagtatgggggccccaatg
gaggaacaatgtcagattgcaagacatccctatattccctgggagacat
tttacagggaagtctcgtttctcttttattattacttttggaatcccgc
ttcttgaaaggagctctcaggtactctgaggtgacatcaccattatgta
ggaggaggctatttcctggagaaggggttcgctt*gattgctggatcaa
atggtagttctacttttagttatttaaggaatctccacactgttttcca
tagtggttgtactagtttgcattcccaccagcagcgcagaggtgttccc
ttttcgccacatccatgccaacatctattatattttgattttttgatta
tggccattcttgcaggagtaaggtgataccgcattgcagttttgatttg
catttccctgatcattagtgatgttgagcgatttttcaagtttgttggc
catttgtatatcttttgagaattgtctattcatgtccttagcccacttt
ttgatggggttctttttgtgtgtgtgtgctaatttgtttgagttccttg
tagattctgcatattagtcctttgttggatgttttaattgtttgttgtg
tgcgttaattgctaagatttttttccca

The RNA junction sequences enabled one to search among the previously unmappable reads for paired ‘junction’ reads that could now specifically map to the junction site with one read, and to one of the fusion gene partners with another read. For most of the validated fusion transcripts, the corresponding junction reads were able to be identified, and this is shown in Table 2 along with the supporting paired chimeric reads.

TABLE 2
Paired chimeric reads and junction reads supporting fusion
transcripts from each patient sample. The number of paired chimeric reads and junction reads
obtained for each of the 15 fusion transcripts contributed by each sequenced sample is shown.
For example, “7 + 19” indicates 7 paired chimeric reads and nineteen junction reads
for the fusion transcript FAM19A3-LPP in sample S3.

| Fusion | | | | | | | | Ovary | Fallopian | Total |
|---|---|---|---|---|---|---|---|---|---|---|
| Transcript | S3 | S4 | S5 | S6 | S10 | S11 | S13 | pool | tube pool | reads |
| CDKN2D- | 0 | 0 | 3 + 7 | 0 | 0 | 0 | 0 | 0 | 0 | 10 |
| WDFY2 | | | | | | | | | | |
| TMEM66- | 0 | 0 | 0 | 6 + 5 | 0 | 0 | 0 | 0 | 0 | 11 |
| MSRB3 | | | | | | | | | | |
| FAM19A3- | 7 + 19 | 0 | 0 | 0 | 0 | 0 | 0 | 0 | 0 | 26 |
| LPP | | | | | | | | | | |
| RFX2- | 0 | 0 | 8 + 3 | 0 | 0 | 0 | 0 | 0 | 0 | 11 |
| CCDC94 | | | | | | | | | | |
| NR2F6- | 0 | 3 + 7 | 0 | 0 | 0 | 0 | 0 | 0 | 0 | 10 |
| MAST3 | | | | | | | | | | |
| WDFY2- | 0 | 0 | 5 + 10 | 0 | 0 | 0 | 0 | 0 | 0 | 15 |
| S1PR5 | | | | | | | | | | |
| CRTAC1- | 0 | 11 + 20 | 0 | 0 | 0 | 0 | 0 | 0 | 0 | 31 |
| GOLGA7B | | | | | | | | | | |
| LAMC2- | 9 + 0 | 4 + 0 | 4 + 0 | 0 | 1 + 0 | 0 | 0 | 0 | 1 + 0 | 19 |
| NMNAT2 | | | | | | | | | | |
| MAG-CD22 | 0 | 0 | 2 + 0 | 7 + 15 | 0 | 0 | 0 | 1 + 0 | 0 | 25 |
| HSP90B1- | 0 | 2 + 0 | 0 | 0 | 0 | 0 | 0 | 1 + 0 | 1 + 0 | 4 |
| C12orf73 | | | | | | | | | | |
| SLC25A29- | 0 | 5 + 6 | 0 | 0 | 0 | 0 | 0 | 0 | 0 | 11 |
| BC014138 | | | | | | | | | | |
| RNF19B- | 2 + 15 | 0 | 0 | 0 | 0 | 0 | 0 | 0 | 0 | 17 |
| BC036308 | | | | | | | | | | |
| C3orf78- | 0 | 3 + 0 | 0 | 0 | 0 | 0 | 0 | 0 | 0 | 3 |
| PBRM1 | | | | | | | | | | |
| RHOBTB2- | 0 | 3 + 1 | 0 | 0 | 0 | 0 | 0 | 0 | 0 | 4 |
| PEBP4 | | | | | | | | | | |
| KRT7- | 4 + 0 | 5 + 2 | 2 + 0 | 0 | 0 | 0 | 0 | 0 | 0 | 13 |
| KRT86 | | | | | | | | | | |

In all, 15 out of a total of 47 putative fusion transcripts chosen for validation were experimentally validated in patient samples used for sequencing. In addition to the HG-SC samples, two endometrioid and one clear cell cancer sample were sequenced which are different subtypes of ovarian carcinoma. Analyses of the output sequences indicated that none of the 15 validated fusion transcripts found in HG-SC samples was present in endometrioid or clear cell cancer samples.

The known chromosomal instability of HG-SC is expected to lead to higher incidence of gene fusions. In the 15 validated fusion transcripts that were identified, 6 are inter-chromosomal recombinant events or long-distance intra-chromosomal recombinant events, indicating that they are the result of major chromosomal rearrangements (Table 3).

TABLE 3
Validated fusion transcripts identified from transcriptome sequencing of HG-SC samples.
Table 3 shows the chromosomal locations, and frequency of occurrence for each fusion
transcript in 28 HG-SC samples, 10 non-cancerous donor ovary samples, and 4 non-
cancerous donor fallopian tube samples as determined by nested RT-PCR. CDKN2D-WDFY2
indicated by “*” was validated in 60 instead of 28 HG-SC samples.

| | | Frequency of | Frequency of | Frequency of |
|---|---|---|---|---|
| | | occurrence among | occurrence among | occurrence among |
| | | 28 High-grade | 10 non-cancer | 4 non-cancer |
| Fusion | | serous cancer | donor | donor fallopian |
| transcript | Type of fusion | samples | ovary samples | tube samples |
| CDKN2D- | Interchromosomal | 12/60 (20%) * | 0 | 0 |
| WDFY2 | (chr 19/13) | | | |
| TMEM66- | Interchromosomal | 5 (18%) | 2 | 0 |
| MSRB3 | (Chr8/12) | | | |
| FAM19A3- | Interchromosomal | 2 (7%) | 0 | 0 |
| LPP | (Chr 1/3) | | | |
| RFX2- | Intrachromosomal | 2 (7%) | 0 | 1 |
| CCDC94 | (Chr 19) | | | |
| NR2F6-MAST3 | Intrachromosomal | 2 (7%) | 1 | 0 |
| | (Chr 19) | | | |
| WDFY2- | Interchromosomal | 1 (3.5%) | 0 | 0 |
| S1PR5 | (Chr 13/19) | | | |

These were chosen for further study as they could represent true fusion genes. The remaining 9 validated fusion transcripts are neighboring gene chimeras. Previously it was found that the majority of fusion transcripts resulting from neighboring genes have no evidence of local genomic rearrangement (Kannan, et al., 2011). These candidates more likely result from transcriptional read-through or local aberrant trans-splicing, and therefore were not selected for current investigation.

### Example 2

### CDKN2D-WDFY2 Occurs in Multiple Cancer Samples

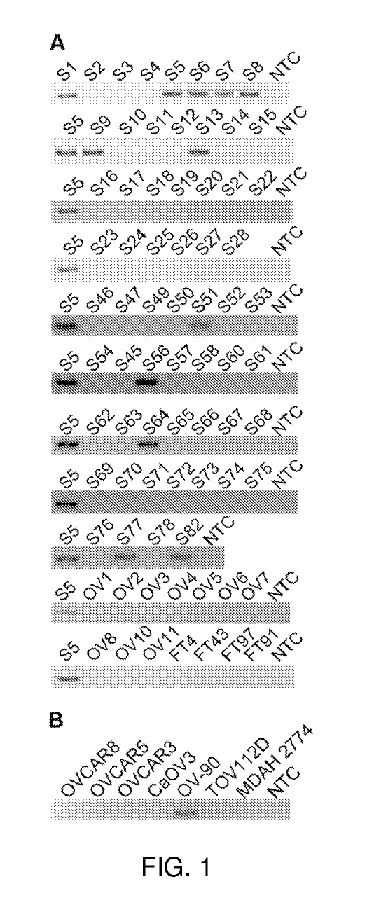

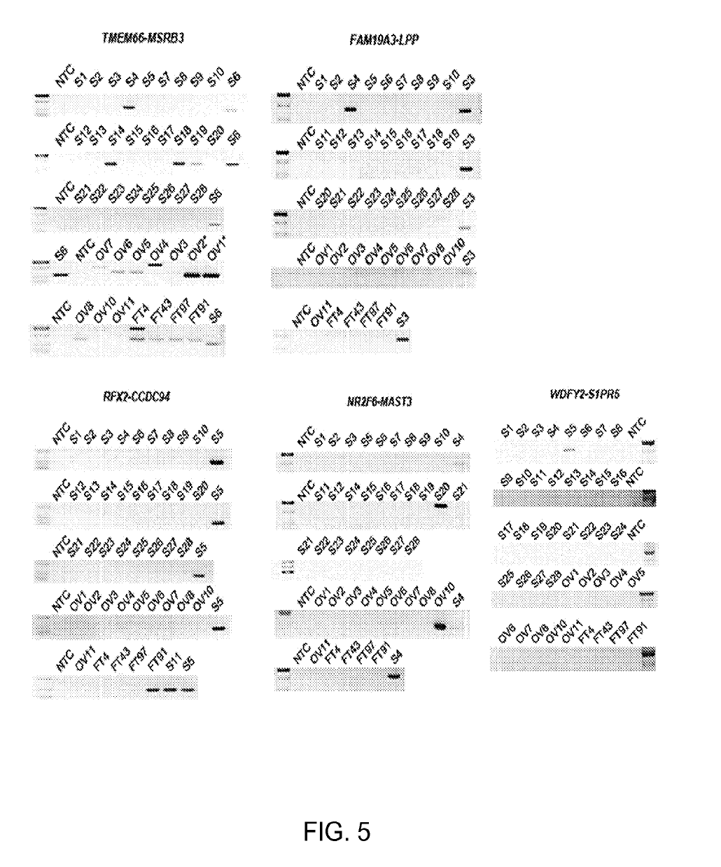

To evaluate the frequency of occurrence of these 6 fusion gene candidates, their expression was tested in a cohort of 28 HG-SC patient samples by nested RT-PCR, which provides highly sensitive and specific amplification. In parallel, nested RT-PCR was also performed on a cohort of 10 non-cancerous donor ovary samples and 4 non-cancerous donor fallopian tube samples. The results indicate that two of the six fusion transcripts were found only among the cancer cohort (Table 3) implying that they are cancer-specific. Among them, CDKN2D-WDFY2 appeared to be a high incidence event. To further evaluate its frequency of occurrence, the cohort size was increased to 60 HG-SC samples and it was found that this fusion transcript was present in 20% (12 out of 60) of HG-SC samples and absent in all non-cancerous ovary and fallopian tube samples, indicating that it is cancer-specific (FIG. 1A). The remaining fusion transcripts displayed lower frequency of occurrence or non-cancerous specific pattern (Table 3). For example, FAM19A3-LPP, RFX2-CCDC94 and NR2F6-MAST3 were present in only 7% of the samples (Table 3 and FIG. 5). TMEM66-MSRB3 was present in 18% of the cancer samples, but it was also expressed in two of the non-cancerous ovary controls.

Because CDKN2D-WDFY2 appeared to be a highly frequent event, it was considered that this fusion transcript may also be present in established HG-SC cell lines. This indeed is the case. By RT-PCR screening of five serous type cell lines (CaOV3, OV-90, OVCAR8, OVCAR5 and OVCAR3), in addition to two endometrioid type cell lines (TOV112D and MDAH 2774), it was found that CDKN2D-WDFY2 fusion transcript is expressed in OV-90, but not others (FIG. 1B). The presence of CDKN2D-WDFY2 in an established HG-SC cell line such as OV-90 further supports the significance of the CDKN2D-WDFY2 fusion in HG-SC.

### Example 3

### CDKN2D-WDFY2 is a Fusion Gene Resulting from a Chromosomal Rearrangement

Because CDKN2D-WDFY2 is an inter-chromosomal fusion event in HG-SC that appears to be cancer-specific and occurs at a frequency that is without precedent in ovarian cancer, this was examined in detail. The identified RNA junction sequence using RT-PCR and Sanger sequencing indicates that exon 1 of CDKN2D is fused to exon 3 of WDFY2 mediated by splicing, and this junction is identical among all patients carrying this fusion transcript. To investigate whether this is the only RNA junction, the transcriptome sequencing data from patient S5 where this fusion transcript was highly expressed was analyzed, and the analysis revealed no other RNA junction. In addition, RT-PCR performed on patient S5 using primer pairs targeting different exons of parental genes also revealed only the same RNA junction. Thus, the results indicate that there is only one RNA junction produced from CDKN2D-WDFY2 fusion, in at least certain embodiments.

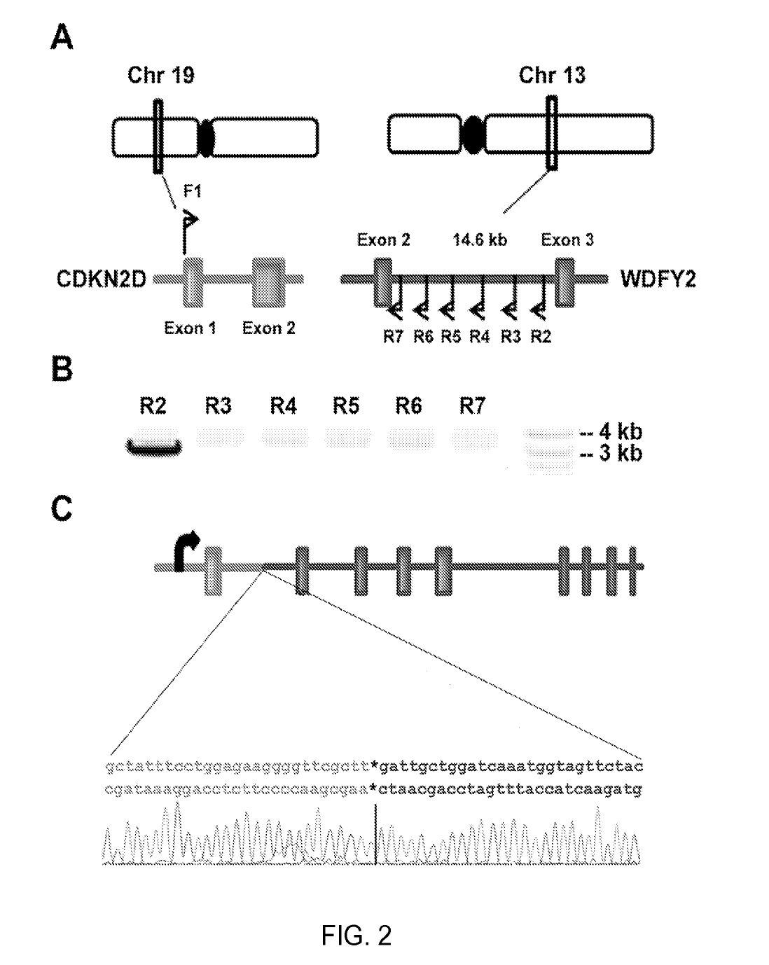

CDKN2D is located in chromosome 19, whereas WDFY2 in chromosome 13. To establish whether this fusion transcript indeed results from chromosomal rearrangement and not from trans-splicing, the genomic breakpoint of CDKN2D-WDFY2 was searched for in the tumor from patient S5. As illustrated in FIG. 2A, several primers were designed to target different locations in intron 2 of WDFY2 (-14 kb long), and these primers were paired with a common primer targeting exon 1 of CDKN2D (CDKN2D intron is comparatively short at ˜1 kb). As shown in FIG. 2B, long-range PCR performed using one particular primer combination on patient tumor genomic DNA led to a single amplified band of approximately 3 kb. Gel purification and Sanger sequencing of this band revealed the precise genomic breakpoint which is located in the intron 679 bp from the end of exon 1 of CDKN2D (chr19:10,678,510) and in the intron 3931 bp upstream from the exon 3 of WDFY2 (chr13:52,245,375) (FIG. 2C and Table 1). This single genomic breakpoint observed in patient S5 provided the direct evidence that CDKN2D-WDFY2 is a fusion gene resulting from a near clonal chromosomal rearrangement. Further sequence analysis of the breakpoint revealed no obvious sequence homology or microhomology around the breakpoint, suggesting that the recombination is possibly mediated by non-homologous end joining (NHEJ) (Lieber, et al., 2010). To answer the question whether the same identified genomic breakpoint occurs in other HG-SC patient samples, genomic DNA was analyzed from 5 patient samples that express the fusion transcript using primers specific for this identified genomic breakpoint. However, none of these samples produced the expected PCR band or unexpected bands. This indicates that locations of genomic breakpoints likely vary among cancer samples and would require different specific primer pairs to probe their locations. This is similar to what was found in prostate cancer in which the genomic breakpoints of TMPRSS2-ERG fusion gene was shown to differ among 35 patient samples, with none of them occurring at the same location (Weier, et al., 2013).

### Example 4

### CDKN2D-WDFY2 Led to the Loss of Cdkn2D Protein Expression and a Gain of a Shortened WDFY2 Protein Isoform

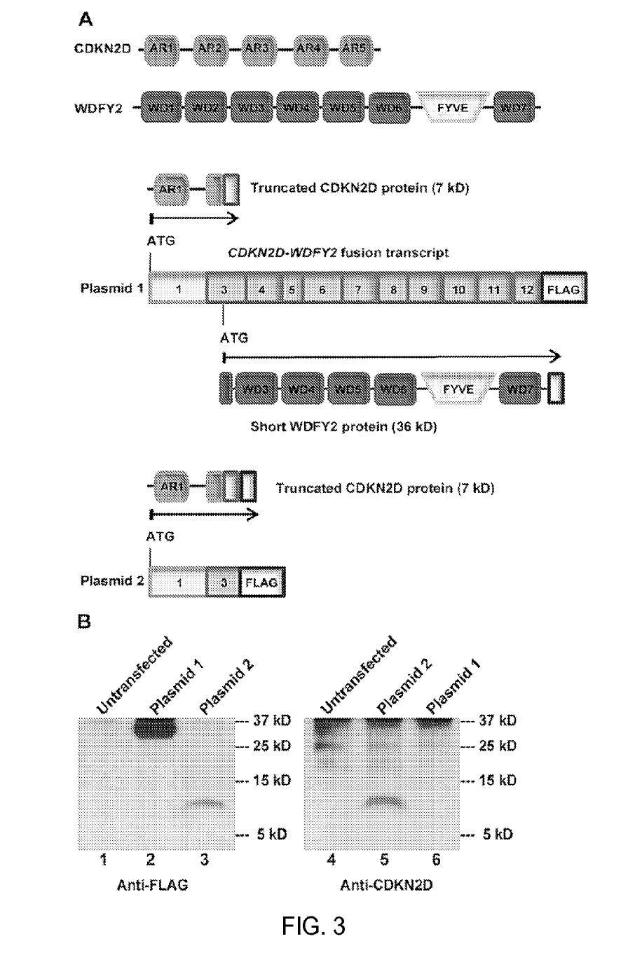

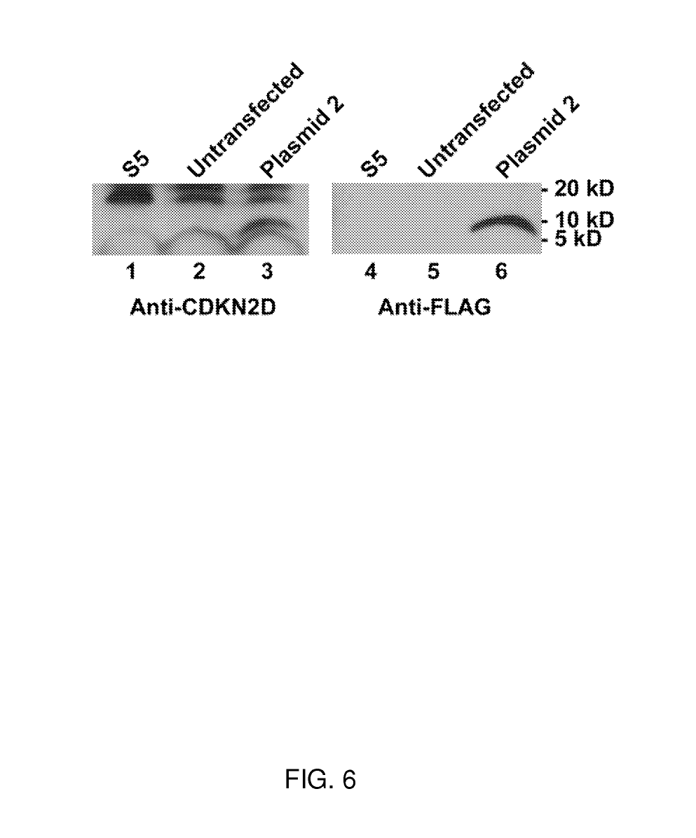

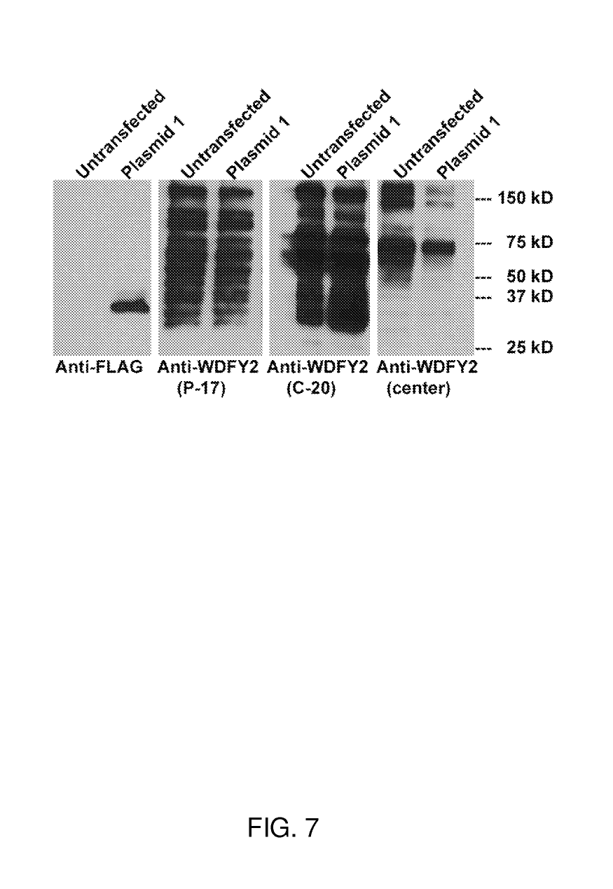

The high rate of occurrence of CDKN2D-WDFY2 among patient tumors suggests that this gene fusion could play a role in oncogenesis. An important yet unanswered question is whether this gene fusion leads to the translation of an aberrant protein. The fusion of exon 1 of CDKN2D to exon 3 of WDFY2 could have two major translational consequences based on the analysis of ‘start’ and ‘stop’ codon in the fusion transcript. The first is a protein of a truncated CDKN2D with the addition of 16 new amino acids from the subsequent out-of-frame WDFY2 sequence, giving rise to a 7 kD protein (FIG. 3A). The second is a short WDFY2 protein resulting from an internal translational initiation site that is in frame with the parental gene stop codon (FIG. 3A). To investigate the translational consequences of the fusion gene, the full-length fusion transcript was cloned (from patient S5) that encompasses both ORFs under the control of CMV promoter (plasmid 1). A FLAG tag was inserted at the C-terminus in frame with WDFY2. A second plasmid was constructed under the control of the same CMV promoter but contained only the ORF for the truncated CDKN2D with a C-terminal FLAG tag (plasmid 2). The truncated CDKN2D protein expressed from plasmid 2 can be detected by both anti-FLAG antibody and a commercial anti-CDKN2D antibody, and the same truncated protein expressed from plasmid 1 by anti-CDKN2D antibody. The short WDFY2 protein expressed from plasmid 1 was visualized by anti-FLAG antibody. Transfection of plasmid 1 and the subsequent Western analysis using anti-FLAG antibody revealed a 36 kDa protein (FIG. 3B, lane 2) that is absent in the untransfected cells (FIG. 3B, lane 1), indicating that this short WDFY2 protein is indeed translated and has the predicted size. In contrast, anti-CDKN2D antibody revealed that the truncated CDKN2D ORF is not selected for translation when plasmid 1 is transfected (FIG. 3B, lane 6), even though it is the first ORF encountered by translational machinery in the fusion transcript. This is not due to antibody recognition issues as the commercial antibody used can readily recognize the truncated CDKN2D protein when plasmid 2 containing only this ORF is transfected (FIG. 3B, lane 3 and 5). Consistent with the observation in transfected cells, protein analysis of tissue from patient S5 also failed to reveal the presence of this 7 kD predicted truncated CDKN2D protein (FIG. 6, compare lane 1 to lane 3). However, efforts to identify the short WDFY2 protein in patient S5 were inconclusive. Three commercially available anti-WDFY2 antibodies were tested that potentially could recognize not only full-length WDFY2 but also short WDFY2. Yet, the western blot results on HEK-293T cells overexpressing short WDFY2 (plasmid 1) showed that these antibodies failed to recognize the short isoform (FIG. 7). Due to this limitation, analysis of the presence/absence of endogenous short WDFY2 protein isoform in patient S5 was not able to be performed, although the corresponding fusion transcript is clearly present.

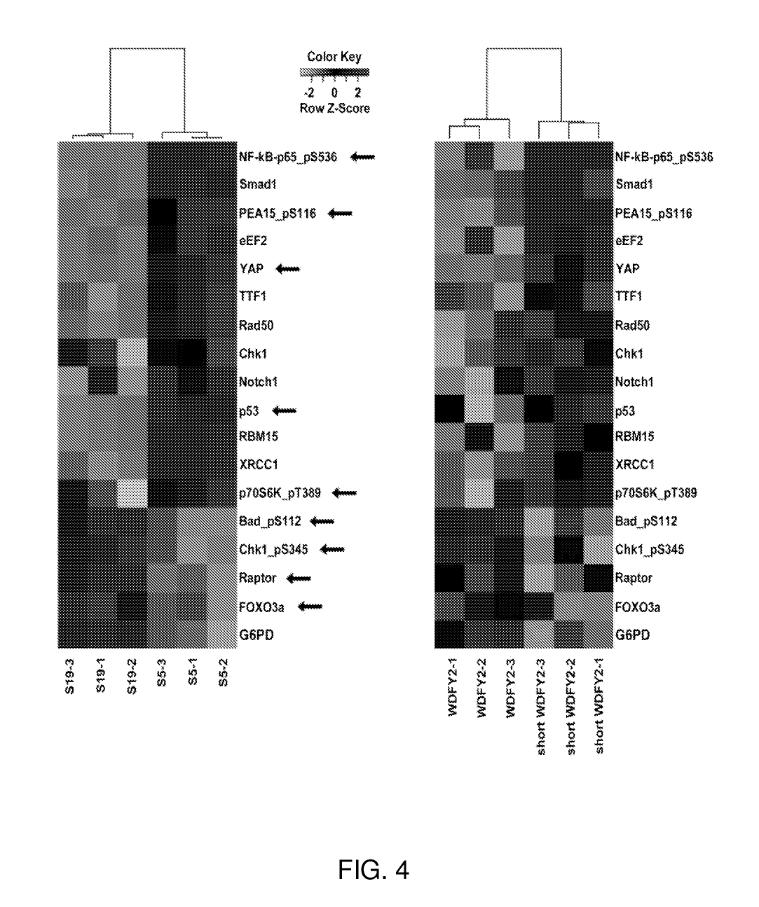

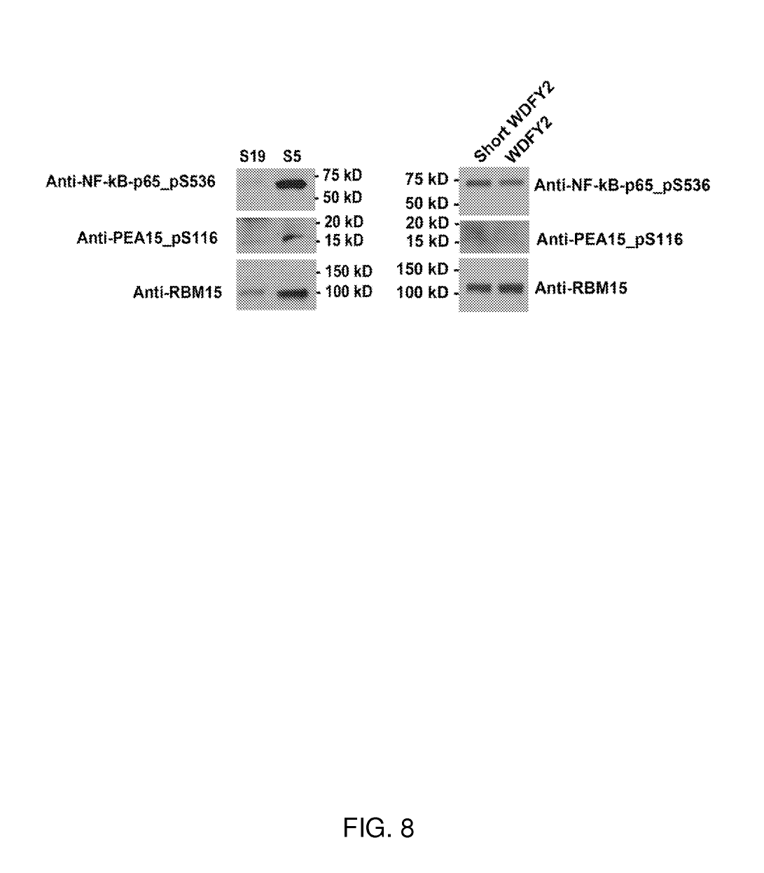

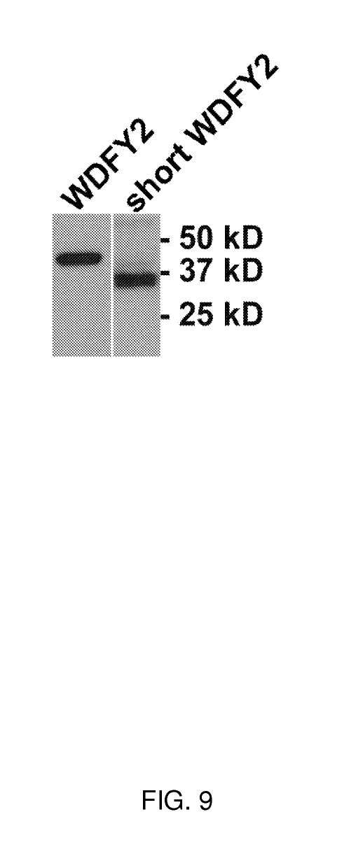

WDFY2 is an endosomal protein with seven WD repeats that is thought to function as a ‘docking station’ that facilitates the interactions between kinases and their substrates. In particular, studies have shown that WDFY2 can bind to AKT and its substrates (Fritzius, et al., 2006; Fritzius, et al., 2008). It was considered that the short WDFY2 protein isoform, which contains only five of the seven WD repeats, may affect the interaction of AKT with its substrates and thus alter downstream signaling. To probe the functional differences of short WDFY2 versus wildtype WDFY2 on signaling pathways, reverse phase protein arrays (RPPA) (Tibes, et al., 2006) were performed, which provides a means to quantitatively assess the levels of 130 cancer-associated proteins using 163 distinct antibodies. The assay was performed on a HG-SC cell line (OVCAR8) transfected with short WDFY2 as compared to wildtype WDFY2, and patient samples S5 (expressing CDKN2D-WDFY2 fusion transcript) as compared to S19 (not expressing the fusion transcript). The analysis revealed 99 proteins that were significantly changed between patients S19 and S5, and 53 proteins between cell lines transfected with wildtype WDFY2 and short WDFY2. To identify those among the significantly changed proteins that could result from the expression of short WDFY2, those proteins that are altered in the same manner in both transfected cell lines and patient samples were sought. This led to a set of 17 proteins whose differential expression levels are shown in FIG. 4. As controls, the expression level of three of these proteins were further confirmed by traditional western analysis (FIG. 8). In addition, it was confirmed that OVCAR8 cells subjected to RPPA analysis have similar levels of transfected wildtype and short WDFY2 (FIG. 9). Therefore, the difference found in RPPA analysis is not due to different protein expression level of wildtype and short WDFY2 in cell line, but likely attributed to their difference in protein activity. However, the levels of endogenous wildtype and short WDFY2 protein isoform in patient S5 and S19 were not able to be confirmed, due to the unavailability of suitable antibodies.

Additional pathway analysis indicates that members of the PI3K/AKT pathway are highly represented in this set of 17 proteins that shows the same alteration in transfected cell lines and patient tissues (hypergeometric test p-value=0.0205). For instance, BAD and FOXO3A, both substrates of AKT, were significantly changed in cell line expressing short versus wildtype WDFY2 and in patient S5 versus S19. This result indicates that the expression of short WDFY2 may alter the PI3K/AKT pathway which may in turn contribute to tumor progression in HG-SC.

### Example 5

### Summary of Certain Embodiments

High-grade serous cancer is characterized by a high degree of heterogeneity among tumors and massive genome rearrangements. This could be related to TP53 as mutations in this gene, seen in almost 96% of high-grade serous tumors, are known to associate with genomic instability. Mutations in common cancer genes such as PTEN, BRCA1, BRCA2, are also present but at much lower prevalence in HG-SC. However, recurrent mutations specific only to HG-SC but not other cancers have been difficult to identify, presumably due to the high heterogeneity among tumors. In contrast, cancers such as chronic myeloid leukemia and prostate cancer can be stratified by a cancer type-specific fusion such as BCR-ABL and TMPRSS2-ERG respectively (Mitelman, et al., 2007). Prior to this study, only one fusion gene, ESRRA-C11orf20, has been found to occur at 15% in HG-SC (Salzman, et al., 2011). This fusion involves two neighboring genes. However, analysis showed that this fusion transcript was absent in sequenced cancer samples presumably due to its lower frequency of occurrence. Moreover, the significance of this fusion for cancer progression is uncertain because it is yet to be established whether it is cancer-specific and translates into a protein product, and present in an established high-grade serous type cell line. The CDKN2D-WDFY2 fusion gene, that was identified by RNA sequencing and validated experimentally in a cohort of 60 patient samples, represents the most frequent cancer-type specific mutation for high-grade serous cancer. Three key features are associated with this recombinant event. First, it is recurrent among 20% of all HG-SC tumors, a significant frequency given the highly heterogeneous nature of this disease. Second, the exact same RNA junction is observed in the fusion transcript across patients suggesting that this mutation leads to a specific aberrant protein function. Third, it is not present in the non-cancerous ovaries or fallopian tubes. All of these features indicate that this gene fusion, alone or in combination with other mutations, could play a role in cancer progression, perhaps by providing survival advantage to cancer cells.

The studies show that the CDKN2D-WDFY2 fusion leads to the loss of translation of wild type CDKN2D and wild type WDFY2, and a gain of a short WDFY2 protein isoform presumably under the control of the CDKN2D promoter. Loss of CDKN2D function can affect both cell cycle regulation as well as DNA repair. CDKN2D (cyclin-dependent kinase inhibitor 2D, or p19 or INK4D) is known to regulate cell-cycle by competing with D-type cyclins for binding to CDK4/6 and to regulate the G1/S transition (Ortega, et al., 2002). CDKN2D also has a distinct role in DNA repair, as its levels are upregulated during genotoxic stress, and the high levels are required for efficient DNA repair (Ceruti, et al., 2005; Scassa, et al., 2007). The loss of functional CDKN2D would mean diminished ability to repair DNA damage that could lead to increased gene mutations and chromosomal recombinations in HG-SC. However, CDKN2D null mice do not develop spontaneous tumors (Zindy, et al., 2000). This indicates that loss of CDKN2D may need to be combined with other common mutations, such as p53 seen in 96% of high-grade serous tumors, to result in a high degree of DNA damage/genome instability that is the hallmark of HG-SC.

WDFY2 contains seven WD (tryptophan-aspartic acid dipeptide) repeats that are thought to form a circularized beta propeller structure. In addition, it also contains a FYVE domain that binds to PI3P (phosphatidylinositol 3-phosphate) on endosomal membranes (Hayakawa, et al., 2006). The WD repeats have been shown to serve as a docking platform for the interaction of AKT and its substrates (Fritzius, et al., 2006; Fritzius, et al., 2008). RPPA data on patient samples and transfected cell lines, which showed that the gain of a short WDFY2 protein may alter the PI3K/AKT pathway, seems to support the above association of WDFY2 with AKT.

The observation that the CDKN2D-WDFY2 fusion transcript exhibited the same RNA junction in patients carrying this fusion transcript and the absence of any other splice variant indicates that this fusion transcript needs to be made precisely, that is, connecting exon 1 of CDKN2D and exon 3 of WDFY2. The results show that this specific connection, which eliminates the original start codon of WDFY2 in the transcript, may lead to the gain of translation of a short WDFY2 protein isoform. Furthermore, the short isoform is presumably under the control of the CDKN2D promoter, thus its expression could be tightly tuned to cell-cycle. Both the short isoform and the misregulation by a cell-cycle dependent promoter could result in an aberrant WDFY2 function affecting PI3K/AKT pathway. Thus, the loss of wildtype CDKN2D and wildtype WDFY2 in combination with the gain of a misregulated short WDFY2 isoform would explain why the fusion gene occurs in 20% of HG-SC tumors, a significant frequency considering the highly heterogeneous nature of HG-SC. A clear cancer phenotype may manifest itself only when the combined context of p53 mutation, loss of CDKN2D, loss of wildtype WDFY2, and gain of misregulated short WDFY2 are present together.

This fusion gene has several clinical utilities. It is useful in stratification of this disease, i.e. in identifying subtypes of HG-SC patients, thus allowing personalized treatment using tailored therapeutics. Upon identification that in some embodiments it is oncogenic, this short WDFY2 protein serves as a therapeutic target for small molecule drugs. Lastly, CDKN2D-WDFY2 is useful as a clinical biomarker for detection of a substantial fraction of HG-SC, as this specific molecular signature is present in circulating cancer cells or in local body fluids released from tumor mass thus detectable using non-invasive assays, in specific embodiments. A specific molecular signature for detection of ovarian cancer has major clinical implications given that much of mortality in ovarian cancer is due to its late detection.

### Example 6

### Sequences for Chimeric RNA and Fusion Genes

Provided below are exemplary sequences for particular chimeric RNA and fusion genes associated with the disclosure. All junctions are indicated by asterisk (*). Start codons and stop codons are indicated in bold (wherever applicable)

### Chimeric RNAs and Their Junctions

CDKN2D-WDFY2 complete cDNA

(SEQ ID NO: 18)

ATGCTGCTGGAGGAGGTTCGCGCCGGCGACCGGCTGAGTGGGGCGGCGG
CCCGGGGCGACGTGCAGGAGGTGCGCCGCCTTCTGCACCGGGAGCTGGTG
CATCCCGACGCCCTCAACCGCTTCGGCAAGACGGCGCTGCAG*CTCCATG
TTCATGCATGTCTTTTAACCCGGAAACAAGAAGACTGTCCATAGGTCTAG
ACAATGGTACAATCTCAGAGTTTATATTGTCAGAAGATTATAACAAGATG
ACTCCTGTGAAAAACTATCAAGCGCATCAGAGCAGAGTGACGATGATCCT
GTTTGTCCTGGAGCTGGAGTGGGTGCTGAGCACAGGACAGGACAAGCAAT
TTGCCTGGCACTGCTCTGAGAGTGGGCAGCGCCTGGGAGGTTATCGGACC
AGTGCTGTGGCCTCAGGCCTGCAATTTGATGTTGAAACCCGGCATGTGTT
TATCGGTGACCACTCAGGCCAAGTAACAATCCTCAAACTGGAGCAAGAAA
ACTGCACCCTGGTCACAACATTCAGAGGACACACAGGTGGGGTGACCGCT
CTCTGTTGGGACCCAGTCCAGCGGGTGTTGTTCTCAGGCAGTTCAGATCA
CTCTGTCATCATGTGGGACATCGGTGGGAGAAAAGGAACAGCCATCGAGC
TCCAAGGACACAACGACAGAGTCCAGGCCCTCTCCTATGCACAGCACACG
CGACAATTGATCTCCTGTGGCGGTGATGGTGGGATTGTCGTCTGGAACAT
GGACGTGGAGAGGCAGGAGACCCCTGAATGGTTGGACAGTGATTCCTGCC
AAAAGTGTGATCAGCCTTTCTTCTGGAACTTCAAGCAAATGTGGGGCAGT
AAGAAAATTGGTCTAAGACAGCACCACTGCCGCAAGTGTGGGAAGGCCGT
CTGTGGCAAGTGCAGCTCCAAGCGCTCCTCCATCCCCCTGATGGGCTTCG
AGTTTGAAGTGAGGGTCTGTGACAGCTGCCACGAGGCCATCACAGATGAA
GAACGTGCACCCACAGCCACCTTCCATGACAGTAAACATAACATTGTGCA
TGTGCATTTCGATGCAACCAGAGGATGGTTACTGACTTCTGGAACTGACA
AGGTTATTAAGTTGTGGGATATGACCCCAGTCGTGTCTTGA
BCAM-AKT2 complete cDNA

(SEQ ID NO: 19)

ATGGAGCCCCCGGACGCACCGGCCCAGGCGCGCGGGGCCCCGCGGCTGC
TGTTGCTCGCAGTCCTGCTGGCGGCGCACCCAGATGCCCAGGCGGAGGTG
CGCTTGTCTGTACCCCCGCTGGTGGAGGTGATGCGAGGAAAGTCTGTCAT
TCTGGACTGCACCCCTACGGGAACCCACGACCATTATATGCTGGAATGGT
TCCTTACCGACCGCTCGGGAGCTCGCCCCCGCCTAGCCTCGGCTGAGATG
CAGGGCTCTGAGCTCCAGGTCACAATGCACGACACCCGGGGCCGCAGTCC
CCCATACCAGCTGGACTCCCAGGGGCGCCTGGTGCTGGCTGAGGCCCAGG
TGGGCGACGAGCGAGACTACGTGTGCGTGGTGAGGGCAGGGGCGGCAGGC
ACTGCTGAGGCCACTGCGCGGCTCAACGTGTTTGCAAAGCCAGAGGCCAC
TGAGGTCTCCCCCAACAAAGGGACACTGTCTGTGATGGAGGACTCTGCCC
AGGAGATCGCCACCTGCAACAGCCGGAACGGGAACCCGGCCCCCAAGATC
ACGTGGTATCGCAACGGGCAGCGCCTGGAGGTGCCCGTAGAGATGAACCC
AGAGGGCTACATGACCAGCCGCACGGTCCGGGAGGCCTCGGGCCTGCTCT
CCCTCACCAGCACCCTCTACCTGCGGCTCCGCAAGGATGACCGAGACGCC
AGCTTCCACTGTGCTGCCCACTACAGCCTGCCCGAGGGCCGCCACGGCCG
CCTGGACAGCCCCACCTTCCACCTCACCCTGCACTATCCCACGGAGCACG
TGCAGTTCTGGGTGGGCAGCCCGTCCACCCCAGCAGGCTGGGTACGCGAG
GGTGACACTGTCCAGCTGCTCTGCCGGGGGGACGGCAGCCCCAGCCCGGA
GTATACGCTTTTCCGCCTTCAGGATGAGCAGGAGGAAGTGCTGAATGTGA
ATCTCGAGGGGAACTTGACCCTGGAGGGAGTGACCCGGGGCCAGAGCGGG
ACCTATGGCTGCAGAGTGGAGGATTACGACGCGGCAGATGACGTGCAGCT
CTCCAAGACGCTGGAGCTGCGCGTGGCCTATCTGGACCCCCTGGAGCTCA
GCGAGGGGAAGGTGCTTTCCTTACCTCTAAACAGCAGTGCAGTCGTGAAC
TGCTCCGTGCACGGCCTGCCCACCCCTGCCCTACGCTGGACCAAGGACTC
CACTCCCCTGGGCGATGGCCCCATGCTGTCGCTCAGTTCTATCACCTTCG
ATTCCAATGGCACCTACGTATGTGAGGCCTCCCTGCCCACAGTCCCGGTC
CTCAGCCGCACCCAGAACTTCACGCTGCTGGTCCAAGGCTCGCCAGAGCT
AAAGACAGCGGAAATAGAGCCCAAGGCAGATGGCAGCTGGAGGGAAGGAG
ACGAAGTCACACTCATCTGCTCTGCCCGCGGCCATCCAGACCCCAAACTC
AGCTGGAGCCAATTGGGGGGCAGCCCCGCAGAGCCAATCCCCGGACGGCA
GGGTTGGGTGAGCAGCTCTCTGACCCTGAAAGTGACCAGCGCCCTGAGCC
GCGATGGCATCTCCTGTGAAGCCTCCAACCCCCACGGGAACAAGCGCCAT
GTCTTCCACTTCGGCACCGTGAGCCCCCAGACCTCCCAGGCTGGAGTGGC
CGTCATGGCCGTGGCCGTCAGCGTGGGCCTCCTGCTCCTCGTCGTTGCTG
TCTTCTACTGCGTGAGACGCAAAGGGGGCCCCTGCTGCCGCCAGCGGCGG
GAGAAGGGGGCTCC*GGAGGAGTGGATGCGGGCCATCCAGATGGTCGCCA
ACAGCCTCAAGCAGCGGGCCCCAGGCGAGGACCCCATGGACTACAAGTGT
GGCTCCCCCAGTGACTCCTCCACGACTGAGGAGATGGAAGTGGCGGTCAG
CAAGGCACGGGCTAAAGTGACCATGAATGACTTCGACTATCTCAAACTCC
TTGGCAAGGGAACCTTTGGCAAAGTCATCCTGGTGCGGGAGAAGGCCACT
GGCCGCTACTACGCCATGAAGATCCTGCGGAAGGAAGTCATCATTGCCAA
GGATGAAGTCGCTCACACAGTCACCGAGAGCCGGGTCCTCCAGAACACCA
GGCACCCGTTCCTCACTGCGCTGAAGTATGCCTTCCAGACCCACGACCGC
CTGTGCTTTGTGATGGAGTATGCCAACGGGGGTGAGCTGTTCTTCCACCT
GTCCCGGGAGCGTGTCTTCACAGAGGAGCGGGCCCGGTTTTATGGTGCAG
AGATTGTCTCGGCTCTTGAGTACTTGCACTCGCGGGACGTGGTATACCGC
GACATCAAGCTGGAAAACCTCATGCTGGACAAAGATGGCCACATCAAGAT
CACTGACTTTGGCCTCTGCAAAGAGGGCATCAGTGACGGGGCCACCATGA
AAACCTTCTGTGGGACCCCGGAGTACCTGGCGCCTGAGGTGCTGGAGGAC
AATGACTATGGCCGGGCCGTGGACTGGTGGGGGCTGGGTGTGGTCATGTA
CGAGATGATGTGCGGCCGCCTGCCCTTCTACAACCAGGACCACGAGCGCC
TCTTCGAGCTCATCCTCATGGAAGAGATCCGCTTCCCGCGCACGCTCAGC
CCCGAGGCCAAGTCCCTGCTTGCTGGGCTGCTTAAGAAGGACCCCAAGCA
GAGGCTTGGTGGGGGGCCCAGCGATGCCAAGGAGGTCATGGAGCACAGGT
TCTTCCTCAGCATCAACTGGCAGGACGTGGTCCAGAAGAAGCTCCTGCCA
CCCTTCAAACCTCAGGTCACGTCCGAGGTCGACACAAGGTACTTCGATGA
TGAATTTACCGCCCAGTCCATCACAATCACACCCCCTGACCGCTATGACA
GCCTGGGCTTACTGGAGCTGGACCAGCGGACCCACTTCCCCCAGTTCTCC
TACTCGGCCAGCATCCGCGAGGGTACCTGA

### Examples of Identified Genomic Breakpoints in Patient Tumors

BCAM-AKT2 genomic breakpoint

(SEQ ID NO: 20)

CCCCCAATCTCCCCTCAACTCCAATCCATAACCCCCTTCAAACCATCCTC
AACTAAGCTCCTCTCCAGCCCTGGCCACATCCCCATCCTCCCCCAACCTC
CAGCCCCAACACCCATCATCCCCCTGAGCTCACCCTTAACTCCAATTTAT
CCTCCAAGCCTATCTCTCACCATCCAGCCCTCACCTAGCCATT*TAGGAG
CACAGAACTGTATACTGTTCTTCACCTTCCTTAATTTTGAAATTTTTTAT
AAAGGCGTGGTCTTCTTATGTTGTCTAGGCTGGTCTGGAATTCCTGGGCT
CAAGCAGTCCTCCAGCCTCTGCCTCCTAAATTGCAGGGATTACATGAGTG
AGCTACCTTGCCCAT
RFX2-CCDC94 genomic breakpoint

(SEQ ID NO: 21)

AGCAAAATCTTTTAATGAGCAGAGGTTTTAAATTTTGGTGAAATCTAATT
TCTCAAGTTTTTCTTTTATGCTGATTTGTGCATTCTAAGAAACCTTTGTC
TAACCTCCTATGTTCTCTCTGAGAAGTCTTATAGTTTTAGCTTTTGTGTG
TAAGTAAGTTCATGTTCTAACTTGAAATCATTTTTATGTATGGTGTGAGG
TAGGGGTCAAGGTTCATTTATTTTCCCCAATATGGATATTCTGTTGATCC
ATCACCATTTGTTTAAAAGACTTCTTTCTCTGTTGAATTGCCTTGACACC
TTTGTGAAAATCCAGTTGAGGAAGAAAAGAGCTGAGGCTCCATTAGAAAA
AAAAATCAATTGATAATCTAAGTGTGGGTTATATTATTTATATATTGTTG
AATTTGATTTATTAATATTTTAAATTATTTTTGCATATATGTGCATAAGA
GATATTAGTCTAAACACTTTCCTTGTAATATATTTGTTAAGTTTTGGTGT
CAGGGTTTACTGACCTCATAAAATGAGTTGTGAAGTATTCTTTTTTTATT
TTCTGATGGCATTTGTGTAATAATTCCTTTTTCAGTATTTGATAGCATTC
ACCAGTAAAACTATATGTGCCTGAAG*GCTGGAGTGCAGTAGTGCAGTCA
TAGCTCACTGTAGCCTCCATCTCCCGGGCTCAAGCGTTCCTCCTGCCTCA
GCCTTCTGGGTGGCTGGGATCACAGGGACACATCACCATGCCCAGCTAAT
TGTTTTTATTTCTGTAGAGACAGGACCTCGCTATGTTGGACAGGCTGGTT
TTGAACTCCTGGCCTCAAGCGATCCTCCCACCTTAGTCTCCTGAAGTGCT
GGATGACACGTGTGAGCCCCTGTGGTTGGCTGGTGAATGCCTTTGACGCC
GACCACCTCTGAGCCCGCGTGCTGATCCCCTCCTTTCCTGGTGCCTCATC
TCCCCTCCCTCCTTTAGAATCTGAGCTGTAAGGACAGGGACGTGGTGGCC
TCTCCTGTGTCCCCAGGGCTTCCAGCAGGTAGGTTC
NR2F6-MAST3 genomic breakpoint

(SEQ ID NO: 22)

GGGCCTGGAGGCTTTGGAGGGGAGGGCCTGAGTGACCCCTTGATGACTTT
TGTCTCCCTTTCCCGTTATTCTCCCTACCTGCCCCCCCCCACCTGCCCCC
CACCCCATGTCTCTATGGGCACCTACTTGGCCCCTCATCCCCCCTCTTCC
GGCCCTCTCCTCTCTACCCCTCGCTTCCCTTCCTGTCTCTCCAATCTCCT
CTCCTCCCGGCCTTCTGTGCCCCGCCCTCCCCTCCCCCATCTCCTGTCCC
TCCCCCGTTTCCTGCTGCCCGCCCCCCCCCCCCCCGCCTCCCCTCCCGCC
CCCATGTCCCT*CCACGTTAAGATGGTCAGGAAGCCTGGGGACCAGTGTT
AGCAGTGGCCTTCCCTTTGTCCTGCAAATTCACAAATGACTTTACCAATT
TCTGTGGCCT

### Example 7

### Exemplary Materials & Methods

Ethics Statement

All tumor samples and non-cancer samples were collected following procedures approved by IRB at Baylor College of Medicine.

Human High-Grade Serous Ovarian Cancer Samples and Cell Lines

Anonymized ovarian cancer tissue samples were obtained from the Tissue Acquisition and Distribution Core of the Dan L. Duncan Cancer Center and Department of Pathology and Immunology and the Gynecologic Oncology Group under an approved Baylor College of Medicine Institutional Review Board protocol. The patient tissues are all fresh frozen samples. All tumor samples were confirmed to have greater than 80% serous adenocarcinoma prior to processing. RNA was extracted from cancer samples and non-cancerous donor samples using Ribopure kit (Ambion).

OVCAR8 cell line was maintained in RPMI-1640 supplemented with 10% FBS and 1% Penicillin/Streptomycin. HEK-293T was maintained in DMEM supplemented with 10% FBS and 1% Penicillin/Streptomycin.

RNA Processing for Paired-End Transcriptome Sequencing

Total RNA samples with RNA integrity number (RIN) of 8 and higher were used for transcriptome sequencing using Illumina mRNA-seq protocol. Briefly, 5 μg of total RNA was used to isolate mRNA using Sera-mag Magnetic Oligo(dT) beads. mRNA was then fragmented and converted into double-stranded cDNA. Adapters were ligated to the double-stranded cDNA and this library was then size-selected to obtain fragments in the range of 200-500 bp. Finally, PCR amplification was performed to obtain the final cDNA library. 10 nM of the library was then used for sequencing. Sequencing of samples S3, S4, S5, S6, CC2, EC2 and EC4 was performed on the Illumina genome analyzer II (GAIT) at the Center for Cancer Epigenetics Solexa Sequencing Core located in the University of Texas—M.D. Anderson Cancer Center with an output of paired end 75-nucleotide reads. Sequencing of the rest of the samples was performed at the Genomic and RNA Profiling Core at Baylor College of Medicine with an output of paired end 100 nucleotide reads.

Bioinformatic Identification of Gene Fusions

RNA-Seq reads were processed by employing the following filters in order: 1) trimming by base quality score in 5′ to 3′ direction, using 15 as minimum threshold, 2) removing reads smaller than 45 basepairs. Roughly 476 million reads uniquely mappable to the human genome UCSC hg19/NCBI chr37 were obtained. Reads were first mapped to the transcriptome using Pash 3.0 (Coarfa, et al., 2010). Reads pairs mapping to non-overlapping genes with 0 mismatches were preserved as inconsistent reads; reads mapping to the same gene or overlapping genes were discarded from analysis. Reads with at most one end mapping to a gene were further selected, and mapped to the genome using bwa (Li, et al., 2009). Again reads mapping to the same gene or overlapping genes were discarded, whereas reads mapping to two different genes were selected. Inconsistent read pairs derived from either transcriptome or genomic mapping were then combined, and non-overlapping gene pairs with at least two read pairs spanning them were selected as candidate gene fusions. The filters (described above) were then used to reduce false positives and thus the 356 putative fusion transcripts from the 7 serous cancer samples were identified

Identification of Junction Reads

RNA junctions were accurately defined using RT-PCR and Sanger sequencing, and then used as templates to align junction reads. Reads that were earlier unmappable to the genome and transcriptome were aligned to the PCR amplicon. A paired read was considered as a junction read only if it met the following conditions: 1) one read of the paired read mapped to either parental gene of the chimeric RNA. 2) Junction read should overlap with at least six nucleotides of the sequence on either side of the RNA junction. 3) Mismatch tolerance was set at two mismatches, but for the six nucleotides flanking the RNA junction, no mismatches were tolerated.

RT-PCR

1 μg of RNA was used for each reverse transcription reaction. RNA was incubated with Oligo dT and dNTPs and denatured at 65° C. This was followed by the addition of a master-mix containing 1× superscript buffer, 10 mM DTT, 5 mM Magnesium chloride, RNaseOUT and Superscript III reverse transcriptase. Reactions were then incubated at 50° for 50 minutes. Reactions were terminated by incubation at 85° for 5 minutes. cDNA was then treated with RNase-H for 20 minutes at 37°. 1 μl of cDNA was used as template for PCR using the primers listed in Table 4. PCR master mixes included 3% DMSO and PCR was done using a standard three-step protocol with annealing temperature of 56° C. The products of RT-PCR were gel purified and sequenced by Sanger sequencing to identify the exact fusion junctions of the candidate events. A minus RT reaction was also conducted and used as negative control.

TABLE 4
Exemplary primers used in experiments.

| Primers for Validation and | | SEQ |
|---|---|---|
| Nested RT-PCR of fusion | | ID |
| transcripts | Sequence 5′-> 3′ | NO |
| CDKN2D-WDFY2 F5 | CGACATGCTGCTGGAGGAGGT | 23 |
| CDKN2D-WDFY2 F1 | GCAGGAGGTGCGCCGCCTTCT | 24 |
| CDKN2D-WDFY2 R1 | GTACCATTGTCTAGACCTATGGA | 25 |
| TMEM66-MSRB3 F1 | CCCATAGGAAAAGTCATCTGT | 26 |
| TMEM66-MSRB3 R1 | CTGCTTGCTCCTCGGCTTGCA | 27 |
| TMEM66-MSRB3 R2 | GCTTGCATTTGTTTCTGCTGA | 28 |
| FAM19A3-LPP F1 | AATGGACAAGTTTCTCCTTCCCCA | 29 |
| FAM19A3-LPP R1 | CACAGCCTCCATCGTCCTGCA | 30 |
| FAM19A3-LPP F2 | GGAGAGGCGGGACTTATTGGA | 31 |
| RFX2-CCDC94 F1 | CATAGAGACTGTAGCCGTGGA | 32 |
| RFX2-CCDC94 R1 | TTACCTCATCCAGGATGGCGGT | 33 |
| RFX2-CCDC94 F2 | GACCAACACGCAGCAGCCGCT | 34 |
| RFX2-CCDC94 R2 | GCTTTCTGGCTTCCTCCAACA | 35 |
| NR2F6-MAST3 F1 | GCAAGCATTACGGTGTCTTCA | 36 |
| NR2F6-MAST3 R1 | GTTACGCAGGATCAAGTTCTGT | 37 |
| NR2F6-MAST3 F2 | AACCTCAGCTACACCTGCCGGT | 38 |
| NR2F6-MAST3 R2 | CAAAGTCGCTTTCGCATGGCT | 39 |
| WDFY2-S1PR5 F1 | CACAGAAAAGAACTCGGTGATGA | 40 |
| WDFY2-S1PR5 R1 | TCTTTCACAGCGACAGAGTCCA | 41 |
| CRTAC1-GOLGA7B F1 | GTTCCAGGTAAGTGTTCCTGA | 42 |
| CRTAC1-GOLGA7B R1 | GTATGTGTCAACACCTATGGA | 43 |
| LAMC2-NMNAT2 F1 | AGGAGCTGGAGTTTGACACGA | 44 |
| LAMC2-NMNAT2 R1 | CGCAAATTCAAATAAGCCAGT | 45 |
| MAG-CD22 F1 | CCATCCTGATTGCCATCGTCT | 46 |
| MAG-CD22 R1 | CCAGCTCTGGGAGGTATGCCT | 47 |
| HSP90B1-C12orf73 F1 | GTCGAGCAGAGGAGACCATGA | 48 |
| HSP90B1-C12orf73 R1 | ACAGAGCAAGACGAAGATGAAGA | 49 |
| SLC25A29-BC014138 F1 | CTTGATCCCTTGGGGGTGCCTT | 50 |
| SLC25A29-BC014138 R1 | AAGTGTGCAGAGGGCTCAGCA | 51 |
| RNF19B-BC036308 F1 | GGAAATTTTCCCCAAAGACACA | 52 |
| RNF19B-BC036308 R1 | CTCACCATCCTCACCCTGACCA | 53 |
| C3orf78-PBRM1 F1 | ATCCAGTGCTTAGTTCCGTCA | 54 |
| C3orf78-PBRM1 R1 | TGGGCAGGTGAGTGTCATTGA | 55 |
| RHOBTB2-PEBP4 F1 | GGGCTGTTCTCATCCTCGTCT | 56 |
| RHOBTB2-PEBP4 R1 | GCGAAGGCGCTGCCGCGAAGA | 57 |
| KRT7-KRT86 F1 | CTGCGTGAGTACCAGGAACTCA | 58 |
| KRT7-KRT86 R1 | ATACTTGAAGGAAGCGGTATGA | 59 |
| Long range PCR | | |
| CDKN2D-WDFY2 F1 | GCAGGAGGTGCGCCGCCTTCT | 60 |
| CDKN2D-WDFY2 R2 | ACAACACTGCATAGGCTACTCT | 61 |
| CDKN2D-WDFY2 R3 | ACACTGGGTACACTGGTCGGGT | 62 |
| CDKN2D-WDFY2 R4 | GCATTATTTGGCAGGAGACACT | 63 |
| CDKN2D-WDFY2 R5 | TAGGGCTACTGCTCCCAATCT | 64 |
| CDKN2D-WDFY2 R6 | AGAACACAGCCAGGATTCTCA | 65 |
| CDKN2D-WDFY2 R7 | AGACATGCAAAGCCTCAAAGA | 66 |
| Primers for CDKN2D- | | |
| WDFY2-FLAG construct: | | |
| CDKN2D CDS fwd | TCGACATGCTGCTGGAGGAGGT | 67 |
| WDFY2 exon 11 rev | TAACCTTGTCAGTTCCAGAAGT | 68 |
| Updated E-S-N primer | GGCAAAGAATTCATAATTAACCGCGGGCGGCCGCCATGCT | 69 |
| Bam-Stop-FLAG-WDFY2 | GCGGATCCCGTCATCACTTGTCGTCATC | 70 |
| CDS end rev | GTCTTTGTAGTCAGACACGACTGGGGTCA | |
| Primers for Truncated | | |
| CDKN2D-FLAG construct: | | |
| W-C F5 | CGACATGCTGCTGGAGGAGGT | 71 |
| W-C R4 | ACAGTCTTCTTGTTTCCGGGT | 72 |
| Eco-Sac-Not-Kozak- | AAGAATTCCGCGGGCGGCCGCCATGCTGCTGGAGGAGGTTCG | 73 |
| CDKN2D fwd | | |
| Bam-Stop-FLAG WDFY2 | GCGGATCCCGTTACTACTTGTCGTCAT | 74 |
| rev | CGTCTTTGTAGTCTGGACAGTCTTCTTGTTT | |
| Primers for WDFY2-FLAG | | |
| construct: | | |
| WDFY2 CDS fwd | ATGGCGGCGGAGATCCAGCCCA | 75 |
| WDFY2 end rev | TCAAGACACGACTGGGGTCA | 76 |
| Updated E-S-N primer for | GGCAAAGAATTCATAATTAACCGCGGGCGGCCGCCATGGC | 77 |
| WDFY2 | | |
| Bam-Stop-FLAG-WDFY2 | GCGGATCCCGTCATCACTTGTCGTCA | 78 |
| CDS end rev | TCGTCTTTGTAGTCAGACACGACTGGGGTCA | |
| Primers for short WDFY2- | | |
| FLAG construct: | | |
| Updated E-S-N primer for | GGCAAAGAATTCATAATTAACCGCGGGCGGCCGCCATGTCT | 79 |
| short WDFY2 | | |
| Bam-Stop-FLAG-WDFY2 | GCGGATCCCGTCATCACTTGTCGTCA | 80 |
| CDS end rev | TCGTCTTTGTAGTCAGACACGACTGGGGTCA | |

Long Range PCR

Using the primers listed in Table 4, long-range PCR was performed on 200 ng of genomic DNA. LA PCR kit (Takara) was used for these reactions and reactions were performed according to the manufacturer's protocols. Two-step PCR was performed with annealing and extension at 68° C. for 20 minutes. Products were run on gels and then gel purified and sequenced by Sanger sequencing.

Cloning and Transfection

Constructs were made for truncated CDKN2D, CDKN2D-WDFY2, wildtype WDFY2 and short WDFY2. Using the primers listed in Table 4, RT-PCR was performed on patient S5 RNA to generate the products. A C-terminal FLAG tag was added to all PCR products and they were cloned into the vector HDM-luc (Ory, et al., 1996) which has a CMV promoter. HEK-293T cells were transfected with the indicated plasmids using TransIT-293 reagent (Mirus) according to the manufacturer's protocol. For RPPA experiments, OVCAR8 cell line was transfected with either WDFY2-FLAG or short WDFY2-FLAG using the Fugene 6 transfection reagent (Roche).

Protein Extraction and Western Blotting

48 hours after transfection, proteins were extracted using RIPA buffer (Santa cruz biotechnology). Briefly, cells were washed with PBS and then RIPA buffer (supplemented with Sodium vanadate, PMSF and protease inhibitors) was added to cells and lysis was allowed to continue for 5 minutes on ice. Then, cells were scraped and collected in Eppendorf tubes and centrifuged at 8000×g for 10 minutes at 4° C. The supernatant was collected and used in western blotting.

For western blotting, equal amounts of lysates were run on a 4-20% Tris-glycine gel (Bio-rad). Proteins were transferred onto nitrocellulose membrane using CAPS buffer (VWR) at 100 V for 1 hour. Membrane was rinsed with 1× TBS and then blocked with 1× TBST containing 5% nonfat dry milk for 2 hours. This was followed by three washes with 1× TBST. Primary antibodies in blocking buffer were incubated with the membrane overnight at 4° C. Following three washes with 1× TBS/T, membrane was incubated with secondary antibodies in blocking buffer for 2 hours. Finally after three washes, detection reagents (Supersignal West Femto from Thermo Scientific) were incubated with the membrane and then exposed to film.

The following antibodies were used: Anti-FLAG (SIGMA F1804), Anti-CDKN2D (Abcam ab102842), Anti-WDFY2 (P-17 (sc-84659) and C-20 (sc-84658)—both from Santa cruz biotechnology), Anti-WDFY2 (Center from Abgent #AP5783c), Anti-rabbit IgG-HRP (Cell Signaling #7074) and Anti-mouse IgG-HRP (Cell Signaling #7076).

Reverse Phase Protein Array (RPPA)

Reverse phase protein array (RPPA) experiment was performed at the University of Texas MD Anderson Cancer Center RPPA core using the antibodies listed in Table 5.

TABLE 5
Examples of Antibodies used in RPPA experiments.

| | | | |
|---|---|---|---|
| ACC1 | eEF2K | p38_MAPK | Rictor |
| ACC_pS79 | EGFR | p38_pT180_{—} | Rictor_pT1135 |
| | | Y182 | |
| ACVRL1 | EGFR_{—} | JNK_pT183_{—} | S6_pS235_{—} |
| | pY1068 | pY185 | S236 |
| Akt | EGFR_{—} | JNK2 | S6_pS240_{—} |
| | pY1173 | | S244 |
| PRAS40_{—} | elF4E | c-Met_pY1235 | P90RSK |
| pT246 | | | |
| Akt_pS473 | 4E-BP1 | c-Myc | P90RSK_{—} |
| | | | pT359_S363 |
| Akt_pT308 | 4E-BP1_pS65 | MYH11 | p70S6K |
| Annexin_{—} | 4E-BP1_{—} | Myosin IIa | p70S6K_{—} |
| VII | pT37_T46 | pS1943 | pT389 |
| AR | elF4G | NDRG1_pT346 | Raptor |
| Bad_pS112 | HER2 | NF2 | SCD1 |
| Bak | HER2_pY1248 | NF-kB-p65_{—} | SF2 |
| | | pS536 | |
| Bax | HER3 | Notch1 | Smad1 |
| Bcl-2 | HER3_pY1289 | N-Ras | Smad3 |
| Bcl-xL | MIG-6 | Heregulin | Smad4 |
| Bim | ER-alpha | DJ-1 | Src |
| Beclin | ER-alpha_pS118 | PCNA | Src_pY416 |
| Bid | FASN | PDCD4 | Src_pY527 |
| clAP | Fibronectin | PDK1 | STAT3_pY705 |
| B-Raf | FoxM1 | PDK1_pS241 | STAT5-alpha |
| BRCA2 | FOX03a | PEA15 | Stathmin |
| TIGAR | FOXO3a_{—} | PEA15_pS116 | Syk |
| | pS318_S321 | | |
| Caspase7_{—} | mTOR | CD31 | Transglutaminase |
| cleavedD198 | | | |
| Caveolin-1 | mTOR_pS2448 | PR | p53 |
| Cyclin_B1 | G6PD | PI3K-p110-alpha | 53BP1 |
| Cyclin_D1 | Gab2 | PI3K-p85 | TRFC |
| Cyclin_E1 | GAPDH | PKC-pan_{—} | TSC1 |
| | | BetalI_pS660 | |
| CDK1 | GATA3 | AMPK_alpha | Tuberin |
| E-Cadherin | GSK3_pS9 | AMPK_pT172 | Tuberin_pT1462 |
| N-Cadherin | GSK3-alpha- | PKC-alpha | TTF1 |
| | beta_pS21_S9 | | |
| p21 | GSK3-alpha-beta | PKC-alpha_pS657 | VHL |
| p27 | IGFBP2 | PKC-delta_pS664 | TAZ |
| p27_pT157 | INPP4B | PTEN | XRCC1 |
| p27_pT198 | IRS1 | Paxillin | YAP |
| Chk1 | CD49b | Rab11 | YAP_pS127 |
| Chk1_{—} | c-Jun_pS73 | Rab25 | YB-1 |
| pS345 | | | |
| Chk2 | VEGFR2 | Rad50 | YB-1_pS102 |
| Chk2_pT68 | c-Kit | Rad51 | 14-3-3_beta |
| Claudin-7 | Lck | C-Raf | 14-3-3_epsilon |
| Collagen_{—} | MEK1 | C-Raf_pS338 | 14-3-3_zeta |
| VI | | | |
| beta-Catenin | MEK1_pS217_{—} | Rb_pS807_S811 | RBM15 |
| | S221 | | |
| eEF2 | MAPK_pT202_{—} | | |
| | Y204 | | |

Tumor or cell lysates (assayed in triplicate) were two-fold-serial diluted for 5 dilutions (from undiluted to 1:16 dilution). Serial diluted lysates were arrayed on nitrocellulose-coated slides and each slide was probed with a validated primary antibody plus a biotin-conjugated secondary antibody. Only antibodies with a Pearson correlation coefficient between RPPA and western blotting of greater than 0.7 were used in RPPA. The signal obtained was amplified using a Dako Cytomation-catalyzed system and visualized by DAB colorimetric reaction. The slides were scanned, analyzed, and quantified using a customized-software Microvigene to generate spot intensity. Relative protein levels for each sample were determined by interpolation of each dilution curves from the standard curve by utilizing the R software package Supercurve (Neeley, et al., 2012; Neeley, et al., 2009). All the data points were further normalized for protein loading. Antibodies were determined with significant changes between the tested conditions by employing the Mann-Whitney-Wilcoxon test (p<0.05), using the R statistical system. Antibodies were further identified that are significantly changed (p<0.05) and in the same direction between the patient samples S5 and S19 and the cell line samples transfected with either full-length WDFY2 or short WDFY2. Pathway analysis was based on the NCI pathway interaction database (http://pid.nci.nih.gov/search/pathway_landing.shtml?what=graphic&jpg=on&pathway_id=pi3kciaktpathway) and Cell Signaling AKT substrate database (http://www.cellsignal.com/reference/pathway/akt_substrates.html). The enrichment of the PI3K/AKT pathway among the identified 17 protein set was determined using the hypergeometric test. RPPA validation was performed on protein extracts from patient tumors S5 and S19 as well as OVCAR8 overexpressing either wt WDFY2 or short WDFY2 using the following antibodies: PEA15_pS116 (Invitrogen #44-836G), RBM15 (Novus Biologicals #21390002) and NF-kBp65_pS536 (Cell Signaling #3033).

### Example 8

### A Recurrent BCAM-AKT2 Fusion Gene Leads to a Constitutively Activated AKT2 Fusion Kinase in High-Grade Serous Ovarian Carcinoma

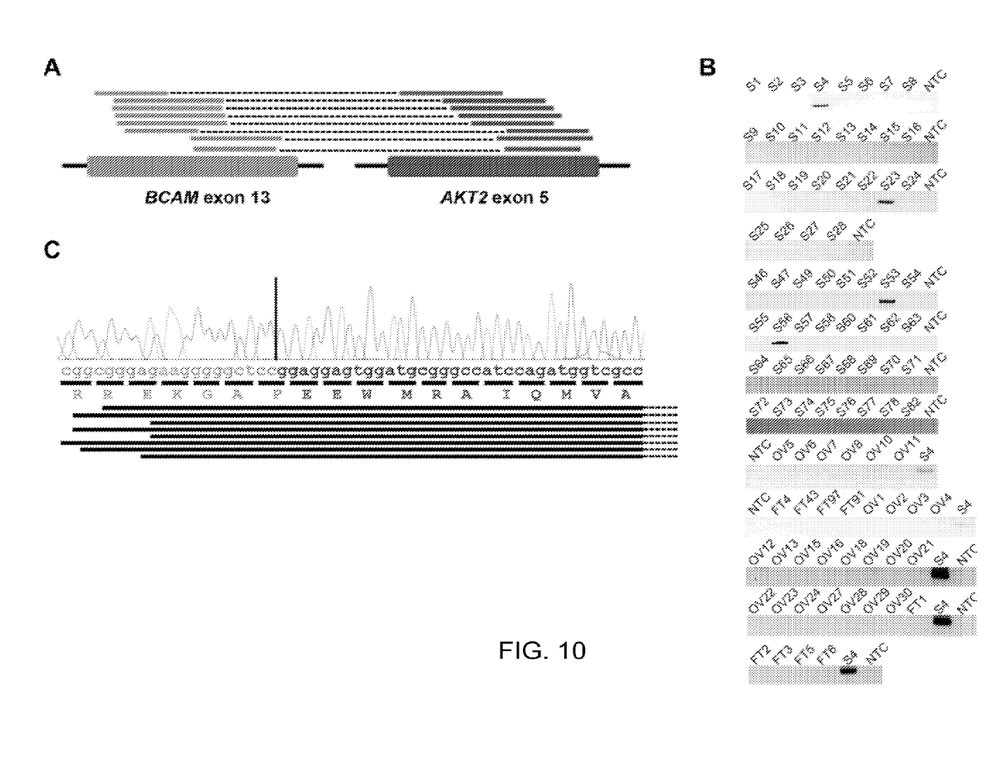

BCAM-AKT2 is a recurrent fusion in HGSC—To examine whether gene fusions involving PI3K members are present in HGSC, we carried out fusion transcript analyses (see SI Materials and Methods) on transcriptome sequencing data from high-throughput sequencing of seven HGSC patient samples (Kannan, et al., 2014). These cancer samples were primary tumors from patients that did not receive neoadjuvant chemotherapy prior to removal of the cancerous ovary. Our analyses uncovered a new fusion transcript between the genes BCAM and AKT2 in one HGSC cancer sample (patient S4, Table 6). This fusion transcript is novel and has not been reported (Kim, et al., 2010; McPherson, et al., 2011; Salzman, et al., 2001; Stransky, et al., 2014). The presence of this fusion transcript in patient S4 is supported by 8 paired ‘chimeric’ reads with one read mapping to BCAM and the other to AKT2 (FIG. 10A). This fusion transcript is absent in the transcriptome sequencing data from pooled non-cancerous ovaries and pooled non-cancerous fallopian tubes (see Materials and Methods). To experimentally validate the presence of this fusion transcript, we performed nested RT-PCR (Table 7 for primers) using a cohort of 60 HGSC patient samples. As shown in FIG. 10B, 4 out of 60 HGSC patient samples gave a distinct band of expected size, indicating an occurrence frequency of ˜7% among this patient population. In contrast, neither the 25 non-cancerous ovary samples nor 9 non-cancerous fallopian tube samples were positive by nested RT-PCR, suggesting that BCAMAKT2 is cancer-specific. All positive RT-PCR bands were gel purified, subjected to Sanger sequencing, and confirmed to harbor the identical BCAM-AKT2 RNA fusion junction, with the 3′ end of BCAM exon 13 (uc002ozu.4) joined to the 5′ end of AKT2 exon 5 (uc002onf.3) by annotated splice sites (FIG. 10C). This indicates that the RNA junction of this fusion transcript is the result of splicing. The identified RNA junction sequence enabled us to search among previously unmapped reads for paired junctionspanning' reads in which one read spans the RNA junction and another read maps to one of the fusion gene partners. We were able to identify 8 corresponding paired junction-spanning reads in patient S4 which further support the presence of the BCAMAKT2 fusion transcript (FIG. 10C, Table 6). No other type of RNA junction was observed in our transcriptome sequencing data or from RT-PCR assays, suggesting that the RNA junction is precisely specified and would lead to a single specified fusion protein.

TABLE 6
Chimeric and junction reads for BCAM-AKT2 from patient
sample S4.

| | | SEQ | | SEQ |
|---|---|---|---|---|
| | | ID | | ID |
| | Read 1 | NO.: | Read 2 | NO.: |
| Chimeric | CGTTGCTGTCTTCTACTGCGT | 81 | CACTGGGGGAGCCACACTTGTA | 82 |
| | GAGACGCAAAGGGGGCCCCT | | GTCCATGGGGTCCTCGCCTGG | |
| | GCTGCCGCCAGCGGCGGG | | GGCCCGC | |
| | | | TGCTTGAGGCTGTTGGCGACCA | |
| | | | TCT | |
| Chimeric | CCAGGCTGGAGTGGCCGTCA | 83 | CTGGGGGAGCCACACTTGTAGT | 84 |
| | TGGCCGTGGCCGTCAGCGTG | | CCATGGGGTCCTCGCCTGGGG | |
| | GGCCTCCTG | | CCCGCTG | |
| | CTCCTCGTCGTTGCTGTCTTC | | CTTGAGGCTGTTGGCGACCATC | |
| | TACTG | | TGG | |
| Chimeric | CTGGAGTGGCCGTCATGGCC | 85 | GGGGTCCTCGCCTGGGGCCCG | 86 |
| | GTGGCCGTCAGCGTGGGCCT | | CTGCTTGAGGCTGTTGGCGACC | |
| | CCTGCTCCT | | ATCTGGA | |
| | CGTCGTTGCTGTCTTCTACTG | | TGGCCCGCATCCACTCCTCCG | |
| | CGTGA | | | |
| Chimeric | GGCCGTCATGGCCGTGGCCG | 87 | GGGTCCTCGCCTGGGGCCCGC | 88 |
| | TCAGCGTGGGCCTCCTGCTC | | TGCTTGAGGCTGTTGGCGACCA | |
| | CTCGTC | | TCTGGAT | |
| | | | GGCCCGC | |
| Chimeric | CTCGTCGTTGCTGTCTTCTAC | 89 | TTTAGCCCGTGCCTTGCTGACC | 90 |
| | TGCGTGAGACGCAAAGGGGG | | GCCACTTCCATCTCCTCAGTCG | |
| | CCCCTGCTG | | TGGAGGA | |
| | CCGCCAGCGGCGGG | | GTCACTGGGGGAGCCACACTT | |
| | | | GTA | |
| Chimeric | CAGACCTCCCAGGCTGGAGT | 91 | AGCCACACTTGTAGTCCATGGG | 92 |
| | GGCCGTCATGGCCGTGGCCG | | GTCCTCGCCTGGGGCCCGCTG | |
| | TCAGCG | | CTTGAGGC | |
| | | | TGTTGGCGACCATCTGGATGGC | |
| | | | CC | |
| Chimeric | GCTGGAGTGGCCGTCATGGC | 93 | GGGGGAGCCACACTTGTAGTC | 94 |
| | CGTGGCCGTCAGCGTGGGCC | | CATGGGGTCCTCGCCTGGGGC | |
| | TCCTGCTCC | | CCGCTGCT | |
| | TCGTCGTTGCTGTCTTCTACT | | TGAGGCTGTTGGCGACCATCTG | |
| | GCGTG | | GAT | |
| Chimeric | CAGGCTGGAGTGGCCGTCAT | 95 | CACACTTGTAGTCCATGGGGTC | 96 |
| | GGCCGTGGCCGTCAGCGTGG | | CTCGCCTGGGGCCCGCTGCTT | |
| | GCCTCCTGC | | GAGGCTGT | |
| | TCCTCGTCGTTGCTGTCTTCT | | TGGCGACCATCTGG | |
| | ACTGC | | | |
| Junction | CAAAGGTTCCCTTGCCAAGGA | 97 | CGGCGGGAGAAGGGGGCTCCG | 98 |
| | GTTTGAGATAGTCGAAGTCAT | | GAGGAGTGGATGCGGGCCATC | |
| | TCATGGTCA | | CAGATGG | |
| | CTTTAGCCCGTGCCTTGCTGA | | TCGCCAACAGCCTCAAGCAGC | |
| | CCG | | GGGCC | |
| Junction | CCGTGAGCCCCCAGACCTCC | 99 | CTTGAGGCTGTTGGCGACCATC | 100 |
| | CAGGCTGGAGTGGCCGTCAT | | TGGATGGCCCGCATCCACTCCT | |
| | GGCCGTGGC | | CCGGAGC | |
| | CGTCAGCGTGGGCCTCCTGC | | CCCCTTCTCCCGCC | |
| | TCCTC | | | |
| Junction | CGTGGGCCTCCTGCTCCTCG | 101 | GCTTGAGGCTGTTGGCGACCAT | 102 |
| | TCGTTGCTGTCTTCTACTGCG | | CTGGATGGCCCGCATCCACTCC | |
| | TGAGACGCA | | TCCGGAG | |
| | AAGGGGGCCCC | | CCCCCCT | |
| Junction | CGGGAACAAGCGCCATGTCT | 103 | CTTGAGGCTGTTGGCGACCATC | 104 |
| | TCCACTTCGGCACCGTGAGC | | TGGATGGCCCGCATCCACTCCT | |
| | CCCCAGA | | CCGGAGC | |
| | | | CCCC | |
| Junction | CGCCATGTCTTCCACTTCGGC | 105 | CCGCTGCTTGAGGCTGTTGGC | 106 |
| | ACCGTGAGCCCCCAGACCTC | | GACCATCTGGATGGCCCGCATC | |
| | CCAGGCTGG | | CACTCCTC | |
| | AGTGGCCGTCATGGCCGTGG | | CGGAGCCCCCTTCTCCCGCC | |
| | CCGTC | | | |
| Junction | AGCGTGGGCCTCCTGCTCCT | 107 | CTCGCCTGGGGCCCGCTGCTT | 108 |
| | CGTCGTTGCTGTCTTCTACTG | | GAGGCTGTTGGCGACCATCTG | |
| | CGTGAGACG | | GATGGCCC | |
| | CAAAGGG | | GCATCCACTCCTCCGGAGCCC | |
| | | | CC | |
| Junction | CTCCCAGGCTGGAGTGGCCG | 109 | CGCTGCTTGAGGCTGTTGGCG | 110 |
| | TCATGGCCGTGGCCGTCAGC | | ACCATCTGGATGGCCCGCATCC | |
| | GTGGGCCTC | | ACTCCTCC | |
| | CTGCTCCTCGTCGTTGCT | | GGAGCCCCCTTCTCCCGCC | |
| Junction | GGAACAAGCGCCATGTCTTC | 111 | CTGGGGCCCGCTGCTTGAGGC | 112 |
| | CACTTCGGCACCGTGAGCCC | | TGTTGGCGACCATCTGGATGGC | |
| | CCAGACCTCC | | CCGCATC | |
| | CAGGCTGGAGTGGCCGTCAT | | CACTCCTCCGGAGCCCCCTTCT | |
| | GGCCG | | CCC | |

TABLE 7
Primers used in this study

| | | SEQ ID |
|---|---|---|
| Primers | Sequence 5′ --> 3′ | NO.: |
| Nested RT-PCR of | | |
| BCAM-AKT2 | | |
| First round | | |
| BCAM F4 | GTGAGCAGCTCTCTGACCCTGA | 113 |
| AKT2 R4 | CGCACCAGGATGACTTTGCCA | 114 |
| Nested PCR | | |
| BCAM-AKT2 F1 | TGTCTTCTACTGCGTGAGACGCA | 115 |
| BCAM-AKT2 R1 | TCTCCTCAGTCGTGGAGGAGT | 116 |
| Long range PCR | | |
| BCAM-AKT2 F1 | TGTCTTCTACTGCGTGAGACGCA | 117 |
| BCAM-AKT2 R1 | TCTCCTCAGTCGTGGAGGAGT | 118 |
| Control primers | AGCAGCTATTCTGCATGGGTGCA | 119 |
| HARS-ZMAT2 F1 | | |
| Control primers | TTTCTCTCTCTTCCGTGAGCCTC | 120 |
| HARS-ZMAT2 R1 | | |
| Primers for | | |
| BCAM-AKT2- | | |
| FLAG construct: | | |
| 5′ BCAM-EcoRI | CGAATTCGGCCGAGCTGCAGCCCGGGCTCAGTCTCCGCCGCCGC | 121 |
| | CGTGAACATGGAGCCCCCGGACGCA | |
| 3′ AKT2-BamHI | GGATCCTTATCACTTGTCGTCATCGTCTTTGTAGTCGGTACCCTCG | 122 |
| | CGGATGCTGGCCGAGTAGGAGAAC | |
| Primers for AKT2- | | |
| FLAG construct: | | |
| E-S-N kozak AKT2 | GGCAAAGAATTCATAATTAACCGCGGGCGGCCGCCATGAATGAGG | 123 |
| fwd | TGTCTGTCA | |
| 3′ AKT2-BamHI | GGATCCTTATCACTTGTCGTCATCGTCTTTGTAGTCGGTACCCTCG | 124 |
| | CGGATGCTGGCCGAGTAGGAGAAC | |

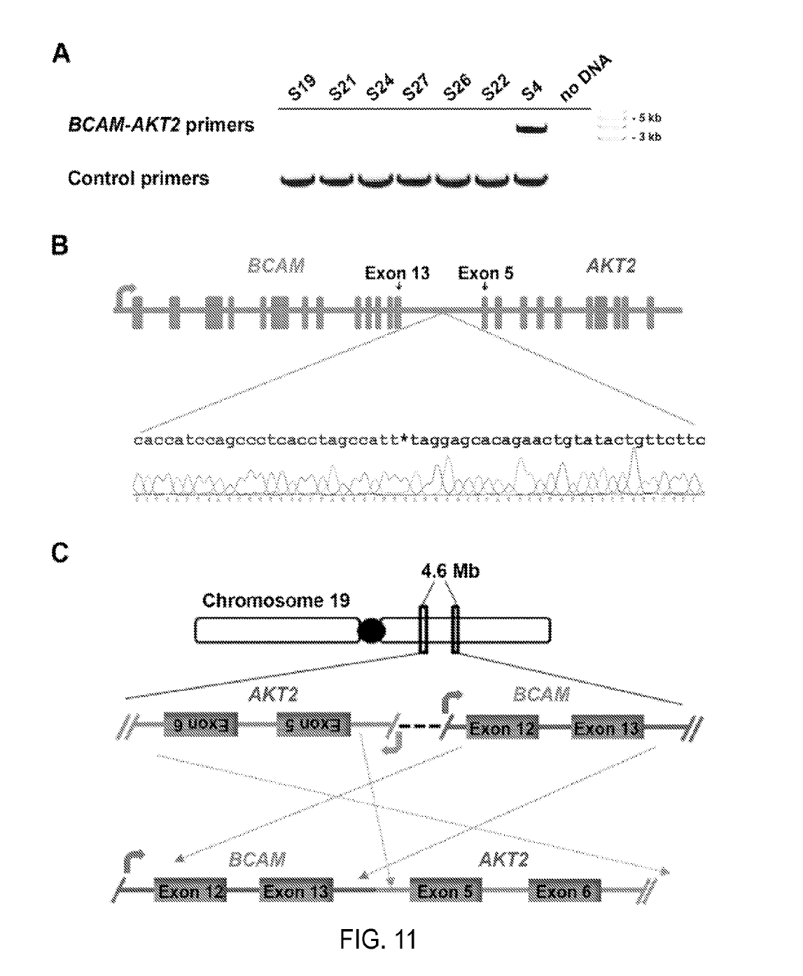

BCAM-AKT2 results from chromosomal rearrangement—BCAM and AKT2 are both located on chromosome 19 and are separated by approximately 4.6 Mb. To check whether the fused BCAM-AKT2 transcript indeed results from chromosomal rearrangement, we employed a primer on BCAM exon 13 paired with another primer on AKT2 exon 5 to amplify from patient S4 genomic DNA. This yielded a single band of ˜3.5 kb which was absent in other patient samples that do not express the fusion transcript (FIG. 11A). Gel purification and Sanger sequencing of this genomic DNA band revealed that the genomic breakpoint is located in the intron 548 bp downstream of exon 13 of BCAM (chr19: 44820273, GRCh38/hg38) and in the intron 2743 bp upstream of exon 5 of AKT2 (chr19: 40245429, GRCh38/hg38) (FIG. 11B, see Example 6). This directly proves that the BCAM-AKT2 transcript results from a gene fusion (FIG. 11C). Further sequence analysis of the breakpoint revealed no obvious sequence homology except a four nucleotide ‘ATTT’ microhomology at the breakpoint, suggesting that the gene fusion may be mediated by non-homologous break repair mechanisms (Lieber, 2010; Zhang, et al., 2010).

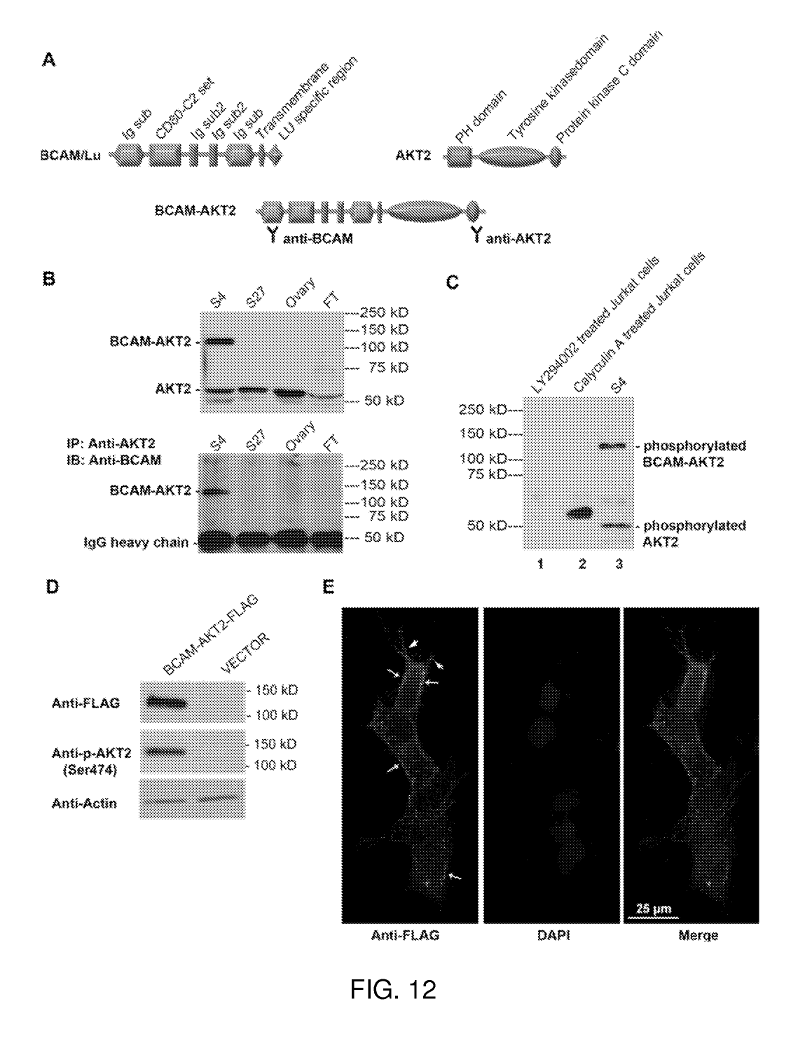

BCAM-AKT2 is translated into an in frame fusion protein in patient tumor—Based on the RNA junction sequence of the fusion transcript, we predicted that translation of BCAM-AKT2 transcript would result in a fusion protein with both BCAM and AKT2 coding regions translated in frame. BCAM gene normally produces two different alternatively spliced RNA isoforms. This fusion retains all domains that define the short protein isoform called BCAM but lacks the last 40 amino acids that define the longer isoform known as Lutheran or Lu (FIG. 12A). The Lu protein isoform is known to localize on the basolateral membrane characteristic of differentiated epithelial cells, while the BCAM isoform, which is overexpressed in ovarian cancers, is localized in a non-polarized manner (El Nemer, et al., 1999; Garinchesa, et al., 1994). The fusion protein is predicted to encode most of AKT2 including its kinase domain (FIG. 12A). However, it lacks 97 amino acids from the N-terminus of AKT2 that constitutes the Pleckstrin homology (PH) domain necessary for targeting AKT2 to the cell membrane where it normally gets activated (Franek, et al., 1995). Since BCAM product is a membrane protein, we predict that the BCAM-AKT2 fusion protein will localize to the membrane.

To confirm the translation of the predicted BCAM-AKT2 fusion protein in HGSC patient, we performed western blot analysis of patient tumor samples and non-cancerous ovary and fallopian tube samples. As shown in FIG. 12B, the AKT2 antibody recognizes a band in patient S4 corresponding to the predicted fusion protein (-110 kDa) which is larger than endogenous AKT2 (˜60 kDa). This 110 kDa band was absent in patient S27 that did not express the fusion transcript, and also lacking in non-cancerous ovary and fallopian tube (FIG. 12B; top panel). To confirm that the 110 kDa band is indeed the BCAM-AKT2 fusion protein, we performed a co-immunoprecipitation assay first by precipitating the target proteins using an anti-AKT2 antibody, followed by western blot analysis using an anti-BCAM antibody. As shown in FIG. 12B bottom panel, the BCAM antibody recognizes the 110 kD band that was precipitated by the AKT2 antibody but does not recognize the precipitated endogenous AKT2 (˜60 kD). This confirmed that the 110 kD band in patient S4 is the BCAM-AKT2 fusion protein and that it is translated in-frame as predicted. Further, western blot analysis using an antibody that specifically detects phosphorylation at serine 474 of AKT2 showed that the AKT2 kinase domain in the fusion protein is phosphorylated (FIG. 12C, lane 3). Together, the results strongly indicate that BCAMAKT2 is translated and phosphorylated in patient tumor.

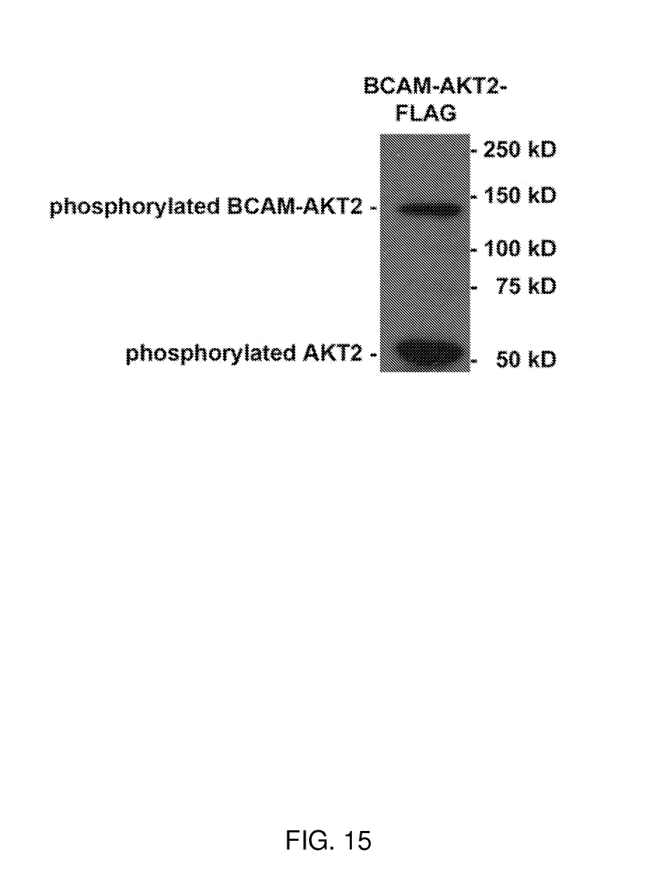

BCAM-AKT2 results in a membrane-associated, constitutively phosphorylated AKT2 fusion kinase—The fact that AKT2 fusion kinase is phosphorylated at serine 474 (FIG. 12C) suggests that loss of the PH domain is compensated by fusion to BCAM, a membrane protein that could re-locate AKT2 to the cell membrane where it gets activated by phosphorylation (Franke, et al., 1995). To confirm this, we set out to generate epitope specific antibodies that could specifically recognize the junction peptide of fusion protein but not parental BCAM or AKT2. However, several monoclonal antibodies that we produced unfortunately failed to recognize the fusion protein specifically. This prevented the most direct confirmation of membrane localization of BCAM-AKT2 fusion protein in patient's tumor, although the fusion protein was clearly translated (FIG. 12B). To circumvent this difficulty, we cloned BCAM-AKT2 from patient S4, FLAG tagged it, and expressed it ectopically in OVCAR8 cells. Western blot analysis confirmed the translation and phosphorylation of BCAM-AKT2 at both serine 474 (FIG. 12D) and threonine 309 of AKT2 (FIG. 15), suggesting that the fusion protein is in the active state. Immunocytochemical imaging showed that the fusion protein is mainly located at the cell membrane (FIG. 12E). Thus, the BCAM-AKT2 fusion leads to a fusion protein that relocates the AKT2 kinase to the cell membrane where it is phosphorylated.

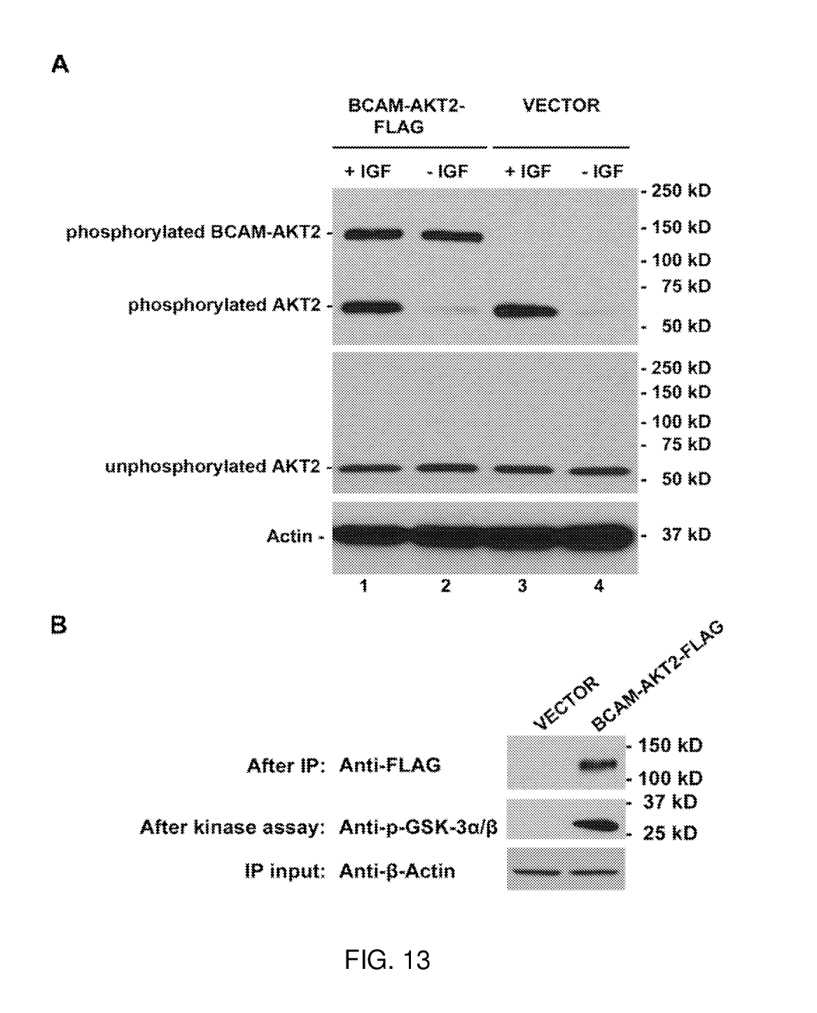

The phosphorylation of AKT2 and thus its activation is known to respond swiftly to extracellular growth stimuli such as insulin like growth factor-1 (IGF-1) (Liu, et al., 1998). To answer the question whether phosphorylation of BCAM-AKT2 fusion protein is also regulated by IGF-1, we serum-deprived transfected OVCAR8 cells overnight so that AKT2 becomes unphosphorylated (Watton & Downward, 1999). Cells were then treated with IGF-1 for 30 minutes to activate AKT2 followed by immediate protein extraction. Western blot analysis using antibody against phosphorylated-AKT2 (serine 474) shows that the endogenous AKT2 responds to IGF-1 treatment swiftly through phosphorylation (FIG. 13A, top panel, lane 3 versus 4). In contrast, BCAM-AKT2 remains phosphorylated regardless of the presence/absence of IGF-1 (FIG. 13A, top panel, lane 1 versus 2). Further, there is no unphosphorylated BCAM-AKT2 detectable (FIG. 13A, middle panel, lane 1 and 2). This result suggests that BCAM-AKT2 is constitutively phosphorylated presumably as a consequence of its membrane localization. To investigate whether the constitutively phosphorylated BCAM-AKT2 still possesses functional kinase activity, we immunoprecipitated BCAM-AKT2 from transfected OVCAR8 cells using anti-FLAG antibody, and incubated the fusion protein with GSK-3, a kinase substrate. As shown in FIG. 13B, incubation with immunoprecipitated BCAM-AKT2 led to efficient phosphorylation of GSK-3, indicating that BCAM-AKT2 is a functional and active kinase.

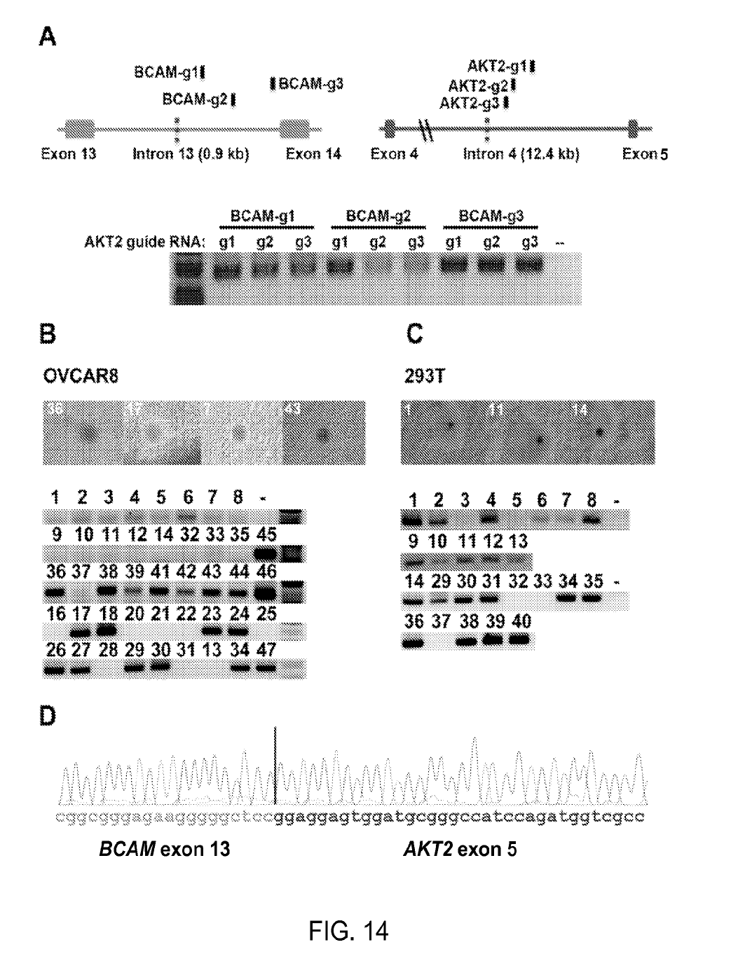

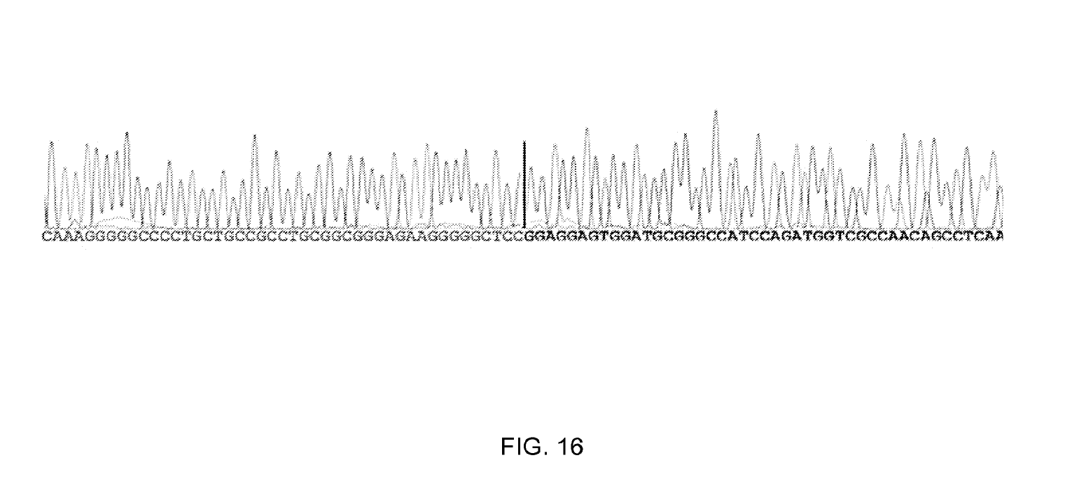

Recreation of the BCAM-AKT2 fusion gene using CRISPR/Cas9 leads to focus formation—To investigate the functional consequences of BCAM-AKT2 fusion gene, we recreated the fusion gene in cells by means of chromosomal translocation induced using the CRISPR/Cas9 system. Unlike commonly used overexpression assays that employ a CMV promoter to express cloned cDNA, the strategy we employed generates a fusion gene with all genomic sequence elements intact, including the endogenous BCAM promoter and the appropriate UTRs and introns. The resulting fusion gene thus would mirror closely the BCAM-AKT2 fusion gene found in patients. To achieve this, we designed three guide RNAs targeting BCAM intron 13 to pair with three guide RNAs targeting AKT2 intron 4 to induce double-stranded breaks at defined sites (FIG. 14A, upper panel). As shown in FIG. 14A lower panel, transfection of nine combinations of paired guide RNAs with Cas9 in HEK-293T cells induced fusion transcript expression to various degrees depending on the combination used. Sanger sequencing of the RT-PCR band from the pair of BCAM-g3/AKT2-g1 confirmed that the induced band is indeed the expected BCAM-AKT2 fusion transcript (FIG. 16).

To assay for an oncogenic phenotype, OVCAR8 and HEK-293T cells were transfected with BCAM-g3/AKT2-g1 along with Cas9 and then plated at a low density to allow individual cells to develop into foci. After 2 weeks, foci were observed sporadically among transfected population but not in the untransfected parental cells. To test if the expression of BCAM-AKT2 is associated with focus formation, 44 prominent foci from transfected OVCAR8 cells and 26 from transfected HEK-293T cells were isolated and cultured. RT-PCR performed on these foci showed that approximately 60% of the isolated foci from OVCAR8 (FIG. 14B) and 80% of the isolated foci from HEK-293T (FIG. 14C) were positive for the BCAM-AKT2 fusion transcript as confirmed by Sanger sequencing of the excised bands (example from an OVCAR8 focus is shown in FIG. 14D). Thus, 60-80% of the isolated foci carry the BCAM-AKT2 translocation, and this is far greater than the expected low rate of long-range chromosome translocation that can be induced by CRISPR/Cas9 system (1-4%) in cells (Torres, et al., 2014). The observed focus formation therefore is not a random event but largely associated with BCAM-AKT2 expression.

Materials & Methods

Human high-grade serous ovarian cancer samples—Sixty anonymous ovarian cancer tissue samples were obtained from the Tissue Acquisition and Distribution Core of the Dan L. Duncan Cancer Center, Department of Pathology & Immunology, and the Gynecologic Oncology Group under an approved Baylor College of Medicine Institutional Review Board protocol. All tumor samples were confirmed to have greater than 80% serous adenocarcinoma prior to processing. Seven of these cancer samples were previously used for high-throughput transcriptome sequencing (Kannan, et al., 2014). We also obtained 25 non-cancerous ovaries and 9 non-cancerous fallopian tubes. One pool of RNA containing 20 non-cancerous ovaries and another pool of RNA containing 6 non-cancerous fallopian tubes were previously used for high-throughput transcriptome sequencing (Kannan, et al., 2014). RNA was extracted from cancer samples and non-cancerous samples using a Ribopure kit (Ambion) and processed for RT-PCR or transcriptome sequencing as described earlier (Kannan, et al., 2014).

Bioinformatic identification of gene fusions—RNA-Seq reads were processed as described in reference (Kannan, et al., 2014). Briefly, this procedure involved the following filters in order: 1) trimming by base quality score in 5′ to 3′ direction, using 15 as minimum threshold, 2) removing reads smaller than 45 basepairs. Reads were first mapped to the transcriptome using Pash 3.0. Reads pairs mapping to non-overlapping genes with 0 mismatches were preserved as inconsistent reads; reads mapping to the same gene or overlapping genes were discarded from analysis. Reads with at most one end mapping to a gene were further selected, and mapped to the genome using bwa. Again reads mapping to the same gene or overlapping genes were discarded, whereas reads mapping to two different genes were selected. Inconsistent read pairs derived from either transcriptome or genomic mapping were then combined, and non-overlapping gene pairs with at least two read pairs spanning them were selected as candidate gene fusions. The analyses were carried out using the high-throughput transcriptome sequencing data of seven cancer samples described previously in (Kannan, et al., 2014). Fusion gene candidates involving PI3K members were chosen for further validation.

Nested RT-PCR—One microgram of total RNA from either the cancerous or non-cancerous sample was used for each reverse transcription reaction. RNA was polyA primed by incubated with Oligo dT and dNTPs, and denatured at 65° C. This was followed by the addition of a master-mix of 20 μl total volume containing: 1× superscript buffer, 10 mM DTT, 5 mM Magnesium chloride, RNaseOUT and Superscript III reverse transcriptase. Reactions were then incubated at 50° C. for 50 minutes. Reactions were terminated by incubation at 85° C. for 5 minutes. cDNA was then treated with RNase-H for 20 minutes at 37° C. Two μl of cDNA from the above reaction was used as template for the first round of PCR using the primers listed in

TABLE 7
PCR master mixes included 3% DMSO and PCR
was performed using the follow temperature:

| | | |
|---|---|---|
| 94° C. -2 minutes | —— | |
| 94° C.- 30 seconds | | |
| 56° C.- 30 seconds | | 38 cycles |
| 72° C.- 1 minute | | |
| 72° C.- 10 minutes | | |

The second round of PCR was also performed using the above PCR conditions. Two μl of the first round PCR product was used as template for the second round PCR using the primers listed in Table 7. As negative control, the PCR was performed without any input cDNA.

Long range PCR—To identify genomic breakpoint, we performed long-range PCR on 200 ng of genomic DNA using LA PCR kit (Takara) with the primers listed in Table 7. Reactions were performed according to the manufacturer's protocols. Two-step PCR was performed with denaturation at 98° C. for 30 seconds followed by annealing and extension both at 68° C. for 20 minutes. Products were run on gels and then gel purified and sequenced by Sanger sequencing.

Cloning and transfection—Plasmid constructs were made to express BCAM-AKT2-FLAG and AKT2-FLAG driven by CMV promoter. Using the primers listed in Table 7, RT-PCR was performed on patient S4 RNA to generate the full-length cDNA products. A C-terminal FLAG tag was added to all PCR products, and they were cloned into the vector HDM (Ory, et al., 1996), which has a CMV promoter. OVCAR8 cell line was obtained from the Matzuk lab and HEK-293T cell line was obtained from ATCC. OVCAR8 cell line was transfected with the indicated plasmids or parental HDM vector using the Fugene 6 transfection reagent (Roche). This was followed by serum deprivation overnight (approximately 16 hours) and then treatment with recombinant human IGF-1 (100 ng/mL) for 30 minutes followed immediately by protein extraction. IGF-1 was purchased from R&D systems (Cat #291-G1-200).

Immunoprecipitation of endogenous BCAM-AKT2 from patient sample—To remove non-specific binding, 50 μl of protein A agarose beads (Cell Signaling 9863) were washed with 500 μl of 1× cell lysis buffer (Cell Signaling 9803) and centrifuged at 6000 rpm for 1 minute at 4° C. and incubated with protein extracts from patient tissues diluted with 1× cell lysis buffer with constant rotation for 1 hour at 4° C. After centrifugation, the supernatant containing unbound proteins was collected. To precipitate the fusion protein, an 1:100 dilution of anti-AKT2 (Cell Signaling 2964) antibody was added to the supernatant, and it was incubated with constant rotation at 4° C. overnight. The next day, freshly prepared protein A agarose beads was added to the supernatant and antibody mixture, and incubated for 3 hours. Then, the beads bound to antibody were collected by centrifugation and washed three times.

After the last wash, most of the buffer was removed, and an equal volume of 2× sample loading buffer was added to the beads. The beads were boiled to detach antibody and proteins from the beads, and after a quick spin, the supernatant was loaded onto a gel for western blot analysis. Anti-BCAM (SIGMA SAB 1100232) antibody was used to detect endogenous BCAM-AKT2 pulled down by anti-AKT2 IP.

Protein extraction of transfected cells and Western blotting—After transfection of expression plasmids, proteins were extracted using NETN buffer (NETN composition: 100 mM NaCl, 1 mM EDTA, 20 mM Tris-HCl pH 7.5 and 0.5% NP-40) at either 24 or 48 hours. Briefly, cells were washed with PBS, NETN buffer (supplemented with SIGMA protease inhibitor cocktail) was added to the cells, and lysis was allowed to proceed for 5 minutes on ice. Cells were scraped and collected in eppendorf tubes and centrifuged at 8000×g for 10 minutes at 4° C. The supernatant was collected and used in western blot analysis.

For western blot analysis, equal amounts of lysates were run on a 4-20% Trisglycine gel (Bio-rad). Proteins were transferred onto nitrocellulose membrane using CAPS buffer (VWR) at 100 V for 1 hour. Membrane was rinsed with 1× TBS and then blocked with 1× TBST containing 5% nonfat dry milk for 2 hours. This was followed by three washes with 1× TBST. Primary antibodies in blocking buffer (5% BSA was used instead of nonfat dry milk in some westerns) were incubated with the membrane overnight at 4° C. Following three washes with 1× TBS/T, membrane was incubated with secondary antibodies in blocking buffer for 2 hours. This was followed by additional three washes. The membrane was then incubated with detection reagents (Supersignal West Femto from Thermo Scientific) and exposed to film. The following antibodies were used for western blot analyses: Anti-FLAG (SIGMA F1804), Anti-BCAM (SIGMA SAB1100232), Anti-AKT2 (Cell Signaling 2964), Anti-phospho AKT2 (serine 474) (SIGMA SAB4300102), Anti-phospho AKT (threonine 308) (Cell Signaling 2965), Anti-rabbit IgG-HRP (Cell Signaling 7074) and Anti-mouse IgG-HRP (Cell Signaling 7076).

FLAG Immunoprecipitation—Protein extracts derived from OVCAR8 cells transfected with either parental HDM plasmid or BCAM-AKT2-FLAG were used for FLAG immunoprecipitation (SIGMA FLAGIPT) as detailed below. Forty ul of beads with conjugated anti-FLAG were washed with 1× wash buffer and centrifuged at 5000×g for 30 seconds, and this was repeated 5 times. The protein extract diluted with NETN buffer was then incubated with the washed beads overnight at 4° C. The next day, beads were centrifuged and the supernatant was removed. Beads were then washed with 1× wash buffer thrice. Elution of immunoprecipitated protein complexes was performed using 3× FLAG peptide as described in FLAG immunoprecipitation kit (SIGMA FLAGIPT) and the eluate was stored at −80° C.

In vitro kinase assay—This assay was performed according to the protocol described in the Nonradioactive AKT Kinase Assay Kit (Cell signaling 9840). Briefly, eluates from the FLAG immunoprecipitation were incubated with 1× kinase buffer, 10 mM ATP and 1 of kinase substrate (GSK-3 fusion protein) at 30° C. for 30 minutes. Then, 6× SDS sample buffer was added to stop the reaction. The samples were then boiled and loaded onto a gel for western blot analysis using anti-phospho-GSK-3a/b (Ser21/9) (Cell Signaling 9327).

Deconvolution fluorescence microscopy and immunofluorescence of transfected cells—OVCAR8 cells were grown on coverslips and then transfected with either parental HDM plasmid or BCAM-AKT2-FLAG. Cells were washed with PBS briefly and then fixed in paraformaldehyde for 10 minutes twice followed by another wash with PBS. Coverslips were then left in triton extraction buffer for 20 minutes on ice. This was followed by two washes with PBS/0.1% NP-40 for 5 minutes each time. Coverslips were then incubated with blocking buffer (10% horse serum/PBS/0.1% NP-40) for one hour. Anti-FLAG antibody (SIGMA F1804) was diluted 1:10,000 in buffer (5% horse serum/PBS/0.1% NP-40) and added to coverslips for one hour. This was followed by three washes with PBS/0.1% NP-40. Alexa Fluor 488 Goat Anti-Mouse IgG (Invitrogen A-11001) was diluted 1:200 in buffer (3% horse serum/PBS/0.1% NP-40) and added to coverslips for one hour. This was followed by three washes and staining with DAPI for 10 minutes. Coverslips were then mounted using Vectashield. Images were taken on a DeltaVision inverted deconvolution/image restoration microscope at the BCM integrated microscopy core facility.

CRISPR/Cas9 guide RNAs for eneration of chromosomal translocation in cell lines—Guide RNA sequence was designed by an online software http://crispr.mit.edu/ (3). For BCAM, the targets of guide RNAs were designed in intron 13 and for AKT2 the targets of guide RNAs were designed in intron 4 (guide RNA sequences are listed in Table 8). The OVCAR8 cell line was transfected with various guide RNAs using the Xtremegene HP DNA transfection reagent (Roche). HEK-293T cell line was transfected with the same guide RNAs using TranslT-293 transfection reagent (Minis). Three days after transfection, cells were split and half the population was used for RNA extraction and RT-PCR while the rest were reseeded and grown. These cells were then plated at a low density of 15,000 cells per 10 cm dish which is commonly used for focus formation assay. Seeded cells were allowed to grow for two weeks with the growth media changed every week. Foci that were observed on these plates were picked and cultured further to obtain enough cells for RNA extraction and RT-PCR.

TABLE 8
Guide RNA Sequences

| | |
|---|---|
| BCAM-g1: TGAAGTTGGCTTCGGGACGG | (SEQ ID NO: 125) |
| BCAM-g2: GTTGGCTTTGGGATCGGTCT | (SEQ ID NO: 126) |
| BCAM-g3: AGCGGGCGGAGGCGTCGTGT | (SEQ ID NO: 127) |
| AKT2-g1: ACGGGGGGTTCAAAATGCGT | (SEQ ID NO: 128) |
| AKT2-g2: GAAATAGAAGTAGTTTCGCG | (SEQ ID NO: 129) |
| AKT2-g3: CAAATCGAAGGCGCTGTCAT | (SEQ ID NO: 130) |

### Example 9

### Examples of Fusion Sequences

Examples of fusion sequences are provided herein, wherein the lowercase denotes 5′ and 3′ UTR, bold letters denote the start and stop codon, and * indicates fusion junction.

MUC1-KRTCAP2 Isoform 1 Complete cDNA:

(SEQ ID NO: 131)

acgctccacctctcaagcagccagcgcctgcctgaatctgttctgccccc
tccccacccatttcaccaccaccATGACACCGGGCACCCAGTCTCCTTTC
TTCCTGCTGCTGCTCCTCACAGTGCTTACAGCTACCACAGCCCCTAAACC
CGCAACAGTTGTTACGGGTTCTGGTCATGCAAGCTCTACCCCAGGTGGAG
AAAAGGAGACTTCGGCTACCCAGAGAAGTTCAGTGCCCAGCTCTACTGAG
AAGAATGCTTTGTCTACTGGGGTCTCTTTCTTTTTCCTGTCTTTTCACAT
TTCAAACCTCCAGTTTAATTCCTCTCTGGAAGATCCCAGCACCGACTACT
ACCAAGAGCTGCAGAGAGACATTTCTGAAATG*AGTGGCAGCAGACGCAC
CAGGTACGAGGAAAGCCTGAGGACCAGGGCGGTGGCCGAGGATGCCCCCT
CGACGCATTATTTCCTCCCTGTCGCAAGAGGGCGCTGCGTCCAGGCACTG
AGAGGAAGAAAGAGGAGAACGCGAGGAGTAGCAAGTCCGCGTGGTGGGTA
CGGGCACCTCGCTGGCGCTCTCCTCCCTCCTGTCCCTGCTGCTCTTTGCT
GGGATGCAGATGTACAGCCGTCAGCTGGCCTCCACCGAGTGGCTCACCAT
CCAGGGCGGCCTGCTTGGTTCGGGTCTCTTCGTGTTCTCGCTCACTGCCT
TCAATAAtctggagaatcttgtctttggcaaaggattccaagcaaagatc
ttccctgagattctcctgtgcctcctgttggctctctttgcatctggcct
catccaccgagtctgtgtcaccacctgcttcatcttctccatggttggtc
tgtactacatcaacaagatctcctccaccctgtaccaggcagcagctcca
gtcctcacaccagccaaggtcacaggcaagagcaagaagagaaactgacc
ctgaatgttcaataaagttgattctttgtaaaaaaaaaaaaaaaaaaaaa
aaaaaaaaaa

MUC1-KRTCAP2 Isoform 1 Protein:

(SEQ ID NO: 132)

MTPGTQSPFFLLLLLTVLTATTAPKPATVVTGSGHASSTPGGEKETSATQ
RSSVPSSTEKNALSTGVSFFFLSFHISNLQFNSSLEDPSTDYYQELQRDI
SEM*SGSRRTRYEESLRTRAVAEDAPSTHYFLPVARGRCVQALRGRKRRT
RGVASPRGGYGHLAGALLPPVPAALCWDADVQPSAGLHRVAHHPGRPAWF
GSLRVLAHCLQ

MUC1-KRTCAP2 Isoform 2 complete cDNA:

(SEQ ID NO: 133)

cgctccacctctcaagcagccagcgcctgcctgaatctgttctgccccct
ccccacccatttcaccaccaccATGACACCGGGCACCCAGTCTCCTTTCT
TCCTGCTGCTGCTCCTCACAGTGCTTACAGCTACCACAGCCCCTAAACCC
GCAACAGTTGTTACGGGTTCTGGTCATGCAAGCTCTACCCCAGGTGGAGA
AAAGGAGACTTCGGCTACCCAGAGAAGTTCAGTGCCCAGCTCTACTGAGA
AGAATGCTTTTAATTCCTCTCTGGAAGATCCCAGCACCGACTACTACCAA
GAGCTGCAGAGAGACATTTCTGAAATGGCTGTCTGTCAGTGCCGCCGAAA
GAACTACGGGCAGCTGGACATCTTTCCAGCCCGGGATACCTACCATCCTA
TGAGCGAGTACCCCACCTACCACACCCATGGGCGCTATGTGCCCCCTAGC
AGTACCGATCGTAGCCCCTATGAGAAG*AGTGGCAGCAGACGCACCAGGT
ACGAGGAAAGCCTGAGGACCAGGGCGGTGGCCGAGGATGCCCCCTCGACG
CATTATTTCCTCCCTGTCGCAAGAGGGCGCTGCGTCCAGGCACTGAGAGG
AAGAAAGAGGAGAACGCGAGGAGTAGCAAGTCCGCGTGGTGGGTACGGGC
ACCTCGCTGGCGCTCTCCTCCCTCCTGTCCCTGCTGCTCTTTGCTGGGAT
GCAGATGTACAGCCGTCAGCTGGCCTCCACCGAGTGGCTCACCATCCAGG
GCGGCCTGCTTGGTTCGGGTCTCTTCGTGTTCTCGCTCACTGCCTTCAAT
AAtctggagaatcttgtctttggcaaaggattccaagcaaagatcttccc
tgagattctcctgtgcctcctgttggctctctttgcatctggcctcatcc
accgagtctgtgtcaccacctgcttcatcttctccatggttggtctgtac
tacatcaacaagatctcctccaccctgtaccaggcagcagctccagtcct
cacaccagccaaggtcacaggcaagagcaagaagagaaactgaccctgaa
tgttcaataaagttgattctttgtaaaaaaaaaaaaaaaaaaaaaaaaaa
aaaaa

MUC1-KRTCAP2 Isoform 2 Protein:

(SEQ ID NO: 134)

MTPGTQSPFFLLLLLTVLTATTAPKPATVVTGSGHASSTPGGEKETSATQ
RSSVPSSTEKNAFNSSLEDPSTDYYQELQRDISEMAVCQCRRKNYGQLDI
FPARDTYHPMSEYPTYHTHGRYVPPSSTDRSPYEK*SGSRRTRYEESLRT
RAVAEDAPSTHYFLPVARGRCVQALRGRKRRTRGVASPRGGYGHLAGALL
PPVPAALCWDADVQPSAGLHRVAHHPGRPAWFGSLRVLAHCLQ

MUC1-KRTCAP2 Isoform 3 Complete cDNA:

(SEQ ID NO: 135)

cgctccacctctcaagcagccagcgcctgcctgaatctgttctgccccct
ccccacccatttcaccaccaccATGACACCGGGCACCCAGTCTCCTTTCT
TCCTGCTGCTGCTCCTCACAGTGCTTACAGCTACCACAGCCCCTAAACCC
GCAACAGTTGTTACGGGTTCTGGTCATGCAAGCTCTACCCCAGGTGGAGA
AAAGGAGACTTCGGCTACCCAGAGAAGTTCAGTGCCCAGCTCTACTGAGA
AGAATGCTTTTAATTCCTCTCTGGAAGATCCCAGCACCGACTACTACCAA
GAGCTGCAGAGAGACATTTCTGAAATGTCTGGGGCTGGGGTGCCAGGCTG
GGGCATCGCGCTGCTGGTGCTGGTCTGTGTTCTGGTTGCGCTGGCCATTG
TCTATCTCATTGCCTTGGCTGTCTGTCAGTGCCGCCGAAAGAACTACGGG
CAGCTGGACATCTTTCCAGCCCGGGATACCTACCATCCTATGAGCGAGTA
CCCCACCTACCACACCCATGGGCGCTATGTGCCCCCTAGCAGTACCGATC
GTAGCCCCTATGAGAAG*AGTGGCAGCAGACGCACCAGGTACGAGGAAAG
CCTGAGGACCAGGGCGGTGGCCGAGGATGCCCCCTCGACGCATTATTTCC
TCCCTGTCGCAAGAGGGCGCTGCGTCCAGGCACTGAGAGGAAGAAAGAGG
AGAACGCGAGGAGTAGCAAGTCCGCGTGGTGGGTACGGGCACCTCGCTGG
CGCTCTCCTCCCTCCTGTCCCTGCTGCTCTTTGCTGGGATGCAGATGTAC
AGCCGTCAGCTGGCCTCCACCGAGTGGCTCACCATCCAGGGCGGCCTGCT
TGGTTCGGGTCTCTTCGTGTTCTCGCTCACTGCCTTCAATAAtctggaga
atcttgtctttggcaaaggattccaagcaaagatcttccctgagattctc
ctgtgcctcctgttggctctctttgcatctggcctcatccaccgagtctg
tgtcaccacctgcttcatcttctccatggttggtctgtactacatcaaca
agatctcctccaccctgtaccaggcagcagctccagtcctcacaccagcc
aaggtcacaggcaagagcaagaagagaaactgaccctgaatgttcaataa
agttgattctttgtaaaaaaaaaaaaaaaaaaaaaaaaaaaaaaa

MUC1-KRTCAP2 Isoform 3 Protein:

(SEQ ID NO: 136)

MTPGTQSPFFLLLLLTVLTATTAPKPATVVTGSGHASSTPGGEKETSATQ
RSSVPSSTEKNAFNSSLEDPSTDYYQELQRDISEMSGAGVPGWGIALLVL
VCVLVALAIVYLIALAVCQCRRKNYGQLDIFPARDTYHPMSEYPTYHTHG
RYVPPSSTDRSPYEK*SGSRRTRYEESLRTRAVAEDAPSTHYFLPVARGR
CVQALRGRKRRTRGVASPRGGYGHLAGALLPPVPAALCWDADVQPSAGLH
RVAHHPGRPAWFGSLRVLAHCLQ

MUC1-KRTCAP2 Isoform 4 Complete cDNA:

(SEQ ID NO: 137)

Gcgcctgcctgaatctgttctgccccctccccacccatttcaccaccacc
atgacaccgggcacccagtctcctttcttcctgctgctgctcctcacagt
gcttacaggtgaggggcacgaggtggggagtgggctgccctgcttaggtg
gtcttcgtggtctttctgtgggttttgctccctggcagatggcaccatga
agttaagctaccacagcccctaaacccgcaacagttgttacgggttctgg
tcatgcaagctctaccccaggtggagaaaaggagacttcggctacccaga
gaagttcagtgcccagctctactgagaagaatgcttttaattcctctctg
gaagatcccagcaccgactactaccaagagctgcagagagacatttctga
aATGTGAgtgatgtgccatttcctttctctgcccagtctggggctggggt
gccaggctggggcatcgcgctgctggtgctggtctgtgttctggttgcgc
tggccattgtctatctcattgccttggctgtctgtcagtgccgccgaaag
aactacgggcagctggacatctttccagcccgggatacctaccatcctat
gagcgagtaccccacctaccacacccatgggcgctatgtgccccctagca
gtaccgatcgtagcccctatgagaag*agtggcagcagacgcaccaggta
cgaggaaagcctgaggaccagggcggtggccgaggatgccccctcgacgc
attatttcctccctgtcgcaagagggcgctgcgtccaggcactgagagga
agaaagaggagaacgcgaggagtagcaagtccgcgtggtgggtacgggca
cctcgctggcgctctcctccctcctgtccctgctgctctttgctgggatg
cagatgtacagccgtcagctggcctccaccgagtggctcaccatccaggg
cggcctgcttggttcgggtctcttcgtgttctcgctcactgccttcaata
atctggagaatcttgtctttggcaaaggattccaagcaaagatcttccct
gagattctcctgtgcctcctgttggctctctttgcatctggcctcatcca
ccgagtctgtgtcaccacctgcttcatcttctccatggttggtctgtact
acatcaacaagatctcctccaccctgtaccaggcagcagctccagtcctc
acaccagccaaggtcacaggcaagagcaagaagagaaactgaccctgaat
gttcaataaagttgattctttgtaaaaaaaaaaaaaaaaaaaaaaaaaaa
aaaa

MUC1-KRTCAP2 Isoform 4 Does Not Result in Any Protein.

MUC1-KRTCAP2 Isoform 5 Complete cDNA:

(SEQ ID NO: 138)

ataattaaccgcgggcggccgccgctccacctctcaagcagccagcgcct
gcctgaatctgttctgccccctccccacccatttcaccaccaccATGACA
CCGGGCACCCAGTCTCCTTTCTTCCTGCTGCTGCTCCTCACAGTGCTTAC
AGTTGTTACGGGTTCTGGTCATGCAAGCTCTACCCCAGGTGGAGAAAAGG
AGACTTCGGCTACCCAGAGAAGTTCAGTGCCCAGCTCTACTGAGAAGAAT
GCTGCTGTCTGTCAGTGCCGCCGAAAGAACTACGGGCAGCTGGACATCTT
TCCAGCCCGGGATACCTACCATCCTATGAGCGAGTACCCCACCTACCACA
CCCATGGGCGCTATGTGCCCCCTAGCAGTACCGATCGTAGCCCCTATGAG
AAG*AGTGGCAGCAGACGCACCAGGTACGAGGAAAGCCTGAGGACCAGGG
CGGTGGCCGAGGATGCCCCCTCGACGCATTATTTCCTCCCTGTCGCAAGA
GGGCGCTGCGTCCAGGCACTGAGAGGAAGAAAGAGGAGAACGCGAGGAGT
AGCAAGTCCGCGTGGTGGGTACGGGCACCTCGCTGGCGCTCTCCTCCCTC
CTGTCCCTGCTGCTCTTTGCTGGGATGCAGATGTACAGCCGTCAGCTGGC
CTCCACCGAGTGGCTCACCATCCAGGGCGGCCTGCTTGGTTCGGGTCTCT
TCGTGTTCTCGCTCACTGCCTTCAATAAtctggagaatcttgtctttggc
aaaggattccaagcaaagatcttccctgagattctcctgtgcctcctgtt
ggctctctttgcatctggcctcatccaccgagtctgtgtcaccacctgct
tcatcttctccatggttggtctgtactacatcaacaagatctcctccacc
ctgtaccaggcagcagctccagtcctcacaccagccaaggtcacaggcaa
gagcaagaagagaaactgaccctgaatgttcaataaagttgattctttgt

MUC1-KRTCAP2 Isoform 5 Protein:

(SEQ ID NO: 139)

MTPGTQSPFFLLLLLTVLTVVTGSGHASSTPGGEKETSATQRSSVPSSTE
KNAAVCQCRRKNYGQLDIFPARDTYHPMSEYPTYHTHGRYVPPSSTDRSP
YEK*SGSRRTRYEESLRTRAVAEDAPSTHYFLPVARGRCVQALRGRKRRT
RGVASPRGGYGHLAGALLPPVPAALCWDADVQPSAGLHRVAHHPGRPAWF
GSLRVLAHCLQ

MUC1-KRTCAP2 Isoform 6 Complete cDNA:

(SEQ ID NO: 140)

Cgctccacctctcaagcagccagcgcctgcctgaatctgttctgccccct
ccccacccatttcaccaccaccatgacaccgggcacccagtctcctttct
tcctgctgctgctcctcacagtgcttacagctaccacagcccctaaaccc
gcaacagttgttacgggttctggtcatgcaagctctaccccaggtggaga
aaaggagacttcggctacccagagaagttcagtgcccagctctactgaga
agaatgctatcccagcaccgactactaccaagagctgcagagagacattt
ctgaaATGTTTTTGCAGATTTATAAACAAGGGGGTTTTCTGGGCCTCTCC
AATATTAAGTTCAGGCCAGGATCTGTGGTGGTACAATTGACTCTGGCCTT
CCGAGAAGGTACCATCAATGTCCACGACGTGGAGACACAGTTCAATCAGT
ATAAAACGGAAGCAGCCTCTCGATATAACCTGACGATCTCAGACGTCAGC
GTGAGTGATGTGCCATTTCCTTTCTCTGCCCAGTCTGGGGCTGGGGTGCC
AGGCTGGGGCATCGCGCTGCTGGTGCTGGTCTGTGTTCTGGTTGCGCTGG
CCATTGTCTATCTCATTGCCTTGGCTGTCTGTCAGTGCCGCCGAAAGAAC
TACGGGCAGCTGGACATCTTTCCAGCCCGGGATACCTACCATCCTATGAG
CGAGTACCCCACCTACCACACCCATGGGCGCTATGTGCCCCCTAGCAGTA
CCGATCGTAGCCCCTATGAGAAG*AGTGGCAGCAGACGCACCAGGTACGA
GGAAAGCCTGAGGACCAGGGCGGTGGCCGAGGATGCCCCCTCGACGCATT
ATTTCCTCCCTGTCGCAAGAGGGCGCTGCGTCCAGGCACTGAGAGGAAGA
AAGAGGAGAACGCGAGGAGTAGCAAGTCCGCGTGGTGGGTACGGGCACCT
CGCTGGCGCTCTCCTCCCTCCTGTCCCTGCTGCTCTTTGCTGGGATGCAG
ATGTACAGCCGTCAGCTGGCCTCCACCGAGTGGCTCACCATCCAGGGCGG
CCTGCTTGGTTCGGGTCTCTTCGTGTTCTCGCTCACTGCCTTCAATAAtc
tggagaatcttgtctttggcaaaggattccaagcaaagatcttccctgag
attctcctgtgcctcctgttggctctctttgcatctggcctcatccaccg
agtctgtgtcaccacctgcttcatcttctccatggttggtctgtactaca
tcaacaagatctcctccaccctgtaccaggcagcagctccagtcctcaca
ccagccaaggtcacaggcaagagcaagaagagaaactgaccctgaatgtt
caataaagttgattctttgtaaaaaaaaaaaaaaaaaaaaaaaaaaaaaa
a

MUC1-KRTCAP2 Isoform 6 Protein:

(SEQ ID NO: 141)

MFLQIYKQGGFLGLSNIKFRPGSVVVQLTLAFREGTINVHDVETQFNQYK
TEAASRYNLTISDVSVSDVPFPFSAQSGAGVPGWGIALLVLVCVLVALAI
VYLIALAVCQCRRKNYGQLDIFPARDTYHPMSEYPTYHTHGRYVPPSSTD
RSPYEK*SGSRRTRYEESLRTRAVAEDAPSTHYFLPVARGRCVQALRGRK
RRTRGVASPRGGYGHLAGALLPPVPAALCWDADVQPSAGLHRVAHHPGRP
AWFGSLRVLAHCLQ

BCAM-AKT2 Fusion Protein:

(SEQ ID NO: 142)

MEPPDAPAQARGAPRLLLLAVLLAAHPDAQAEVRLSVPPLVEVMRGKSVI
LDYTPTGTHDHYMLEWFLTDRSGARPRLASAEMQGSELQVTMHDTRGRSP
PYQLDSQGRLVLAEAQVGDERDYVCVVRAGAAGTAEATARLNVFAKPEAT
EVSPNKGTLSVMEDSAQEIATCNSRNGNPAPKITWYRNGQRLEVPVEMNP
EGYMTSRTVREASGLLSLTSTLYLRLRKDDRDASFHCAAHYSLPEGRHGR
LDSPTFHLTLHYPTEHVQFWVGSPSTPAGWVREGDTVQLLCRGDGSPSPE
YTLFRLQDEQEEVLNVNLEGYLTLEGVTRGQSGTYGCRVEDYDAADDVQL
SKTLELRVAYLDPLELNEGKVLSLPLNSSAVVNCSVHGLPTPALRWTKDS
TPLGDGPMLSLSSITFDSNGTYVCEASLPTVPVLSRTQNFTLLVQGSPGL
KTAEIEPKADGSWREGDEVTLICSARGHPDPKLSWSQLGGSPAEPIPGRQ
GWVSSSLTLKVTSALSRDGISCEASNPHGNKRHVFHFGTVSPQTSQAGVA
VMAVAVSVGLLLLVVAVFYCVRRKGGPCCRQRREKGAP*EEWMRAIQMVA
NSLKQRAPGEDPMDYKCGSPSDSSTTEEMEVAVSKARAKVTMNDFDYLKL
LGKGTFGKVILVREKATGRYYAMKILRKEVIIAKDEVAHTVTESRVLQNT
RHPFLTALKYAFQTHDRLCFVMEYANGGELFFHLSRERVFTEERARFYGA
EIVSALEYLHSRDVVYRDIKLENLMLDKDGHIKITDFGLCKEGISDGATM
KTFCGTPEYLAPEVLEDNDYGRAVDWWGLGVVMYEMMCGRLPFYNQDHER
LFELILMEEIRFPRTLSPEAKSLLAGLLKKDPKQRLGGGPSDAKEVMEHR
FFLSINWQDVVQKKLLPPFKPQVTSEVDTRYFDDEFTAQSITITPPDRYD
SLGLLELDQRTHFPQFSYSASIRE

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

Altomare D A, et al., Oncogene 23(34):5853-5857, 2004.

Andjelkovic M, et al., J Biol Chem 272(50):31515-31524, 1997.

Atherton et al., Biol Reprod. 32(1):155-71, 1985.

Banerji S, et al., Nature 486(7403):405-409, 2012.

Bast & Mills, Cancer Discov 2(1):16-18, 2012.

Bell, et al., Nature 474: 609-615, 2011.

Bellacosa A, et al., Int J Cancer 64(4):280-285, 1995.

Berberian et al., Science. 261(5128):1588-91, 1993.

Brown et al., 1990; Abbondanzo et al., 1990; Allred et al., 1990.

Carpten J D, et al., Nature 448(7152):439-444, 2007.

Ceruti, et al., Oncogene 24: 4065-4080, 2005.

Cheng J Q, et al., Proc Natl Acad Sci USA 89(19):9267-9271, 1992.

Cleary et al., J Immunol. 152(5):2163-71, 1994.

Coarfa, et al., BMC Bioinformatics 11: 572, 2010.

Cong, et al., Science. 339(6121):819-23, 2013.

Cumber et al., J Immunology 149B:120-126, 1992.

De Jager, et al., Semin Nucl Med. 23(2):165-79, 1993.

Dholakia et al., J Biol Chem. 264(1):546-50, 1989.

Doolittle M H and Ben-Zeev O, 1999.

El Nemer W, et al., J Biol Chem 274(45):31903-31908, 1999.

Franke T F, et al., Cell 81(5):727-736, 1995.

Fritzius, et al., Biochem J 399: 9-20, 2006.

Fritzius, et al., EMBO J 27: 1399-1410, 2008.

Garinchesa, Int J Oncol 5(6):1261-1266, 1994.

Garofalo R S, et al., J Clin Invest 112(2):197-208.

Gulbis and Galand, Hum Pathol. 24(12):1271-85, 1993.

Hayakawa, et al., Proc Natl Acad Sci USA 103: 11928-11933, 2006.

Hers, et al., Cell Signal 23(10):1515-1527, 2011.

Honeyman J N, et al., Science 343(6174):1010-1014.

Hwang et al., J Clin Pharmacol. 38(1):60-7, 1998

Jarvius, et al., Molecular & Cellular Proteomics 6 (9): 1500-9, 2007.

Jemal, et al., CA Cancer J Clin 61: 69-90, 2011.

Kang et al., Hybridoma. 17(5):463-70, 1988.

Kannan, et al., PLoS Genet. 10(3):e1004216, 2014.

Kannan, et al., Proc Natl Acad Sci USA 108: 9172-9177, 2011.

Kent, Genome Res 12: 656-664, 2002.

Khatoon et al., Prog Clin Biol Res. 317:801-7, 1989.

Kim P, et al., Nucleic Acids Res 38(Database issue):D81-85, 2010.

King et al., Biochemistry. 28(22):8833-9, 1989.

Kohler et al., Clin Immunol Immunopathol. 52(1):104-16, 1989.

Lee, et al., J Pathol 211: 26-35, 2007.

Lenert et al., Science. 248(4963): 1639-43, 1990.

Li, et al., Bioinformatics 25: 1754-1760, 2009.

Lieber, Annu Rev Biochem 79:181-211, 2010.

Liu, et al., Cancer Res 58(14):2973-2977, 1998.

Liu et al. Cell Mol Biol (Noisy-le-grand). 49(2):209-16, 2003.

Mathiowitz et al., Nature. 386(6623):410-4, 1997.

McPherson, et al., PLoS Comput Biol 7: e1001138, 2011.

Meier, et al., J Biol Chem. 272(48):30491-30497, 1997.

Mende, et al., Oncogene 20(32):4419-4423, 2001.

Micci F, et al., PLoS Biol 12(2):e1001784, 2014.

Mitelman, et al., Nat Rev Cancer 7(4):233-245, 2007.

Neeley, et al., Bioinformatics 25: 1384-1389, 2009.

Neeley, et al., Cancer Inform 11: 77-86, 2012.

Ortega, et al., Biochim Biophys Acta 1602: 73-87, 2002.

Ory, et al., Proc Natl Acad Sci USA 93: 11400-11406, 1996.

O'Shannessy et al., J Immunol Methods. 99(2):153-61, 1987.

Owens & Haley, Biochem Biophys Res Commun. 142(2):964-71, 1987.

Pack et al., Biochem 31:1579-1584, 1992.

Perner, et al., Cancer Res 66: 8337-8341, 2006.

Pothuri, et al., PLoS One 5: e10358, 2010.

Potter & Haley, Methods Enzymol. 91:613-33, 1983

Romero & Bast, Endocrinology 153(4):1593-1602.

Salzman J, et al., PLoS Biol 9(9):e1001156, 2011.

Sasso et al., J Immunol. 142(8):2778-83, 1989.

Scassa, et al., DNA Repair (Amst) 6: 626-638, 2007.

Shokri et al., Clin Exp Immunol. 85(1):20-7, 1991.

Silverman, et al., J Clin Invest. 96(1):416-26, 1995.

Singh D, et al., Science 337(6099):1231-1235.

Söderberg, et al., Nature Methods 3 (12): 995-1000, 2006.

Stransky, Nat Commun 5:4846, 2014.

Tibes, et al., Mol Cancer Ther 5: 2512-2521, 2006.

Tomlins, et al., Science 310(5748):644-648, 2005.

Torres R, et al., Nat Commun 5:3964, 2014.

Watton & Downward, Curr Biol 9(8):433-436, 1999.

Weier, et al., J Pathol 230: 174-183, 2013.

Yuan Z Q, et al., Oncogene 19(19):2324-2330, 2000.

Zhang, Trends Genet 25(7):298-307, 2009.

Zindy, et al., Mol Cell Biol 20: 372-378, 2000.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. As a composition of matter, an isolated chimeric RNA of Table 1 or an isolated polypeptide produced therefrom, or an isolated MUC1-KRTCAP2 chimeric RNA or an isolated polypeptide produced therefrom.

2. The composition of claim 1, wherein the chimeric RNA is CDKN2D-WDFY2.

3. The composition of claim 1, wherein the chimeric RNA is BCAM-AKT2.

4. A substrate comprising polynucleotides attached thereto, said polynucleotides defined as one or more isolated chimeric RNAs of claim 1.

5. The substrate of claim 4, wherein all or greater than 50% , 55%, 60%, 65%, 70%, 75%, 80%, 85%, 90%, 95%, 97%, or 99% of the polynucleotides attached to the substrate are one or more isolated CDKN2D-WDFY2 chimeric RNAs of claim 1.

6. The substrate of claim 4, wherein all or greater than 50% , 55%, 60%, 65%, 70%, 75%, 80%, 85%, 90%, 95%, 97%, or 99% of the polynucleotides attached to the substrate are one or more isolated MUC1-KRTCAP2 chimeric RNAs of claim 1.

7. The substrate of claim 4, wherein all or greater than 50% , 55%, 60%, 65%, 70%, 75%, 80%, 85%, 90%, 95%, 97%, or 99% of the polynucleotides attached to the substrate are one or more isolated BCAM-AKT2 chimeric RNAs of claim 1.

8. A substrate comprising polypeptides attached thereto, said polypeptides defined as one or more isolated gene products of the chimeric RNAs of claim 1.

9. A method of determining a diagnosis, prognosis, risk for, or treatment for ovarian cancer in an individual, comprising the step of assaying a sample from the individual for the presence of a fusion gene that produces the composition of claim 1, assaying a sample from the individual for the presence of the composition of claim 1, or assaying a sample from the individual for the presence of a polypeptide produced from the composition of claim 1.

10. The method of claim 9, wherein assaying for the fusion gene utilizes FISH, long-range PCR, Southern blotting, karyotyping, or a combination thereof.

11. The method of claim 9, wherein assaying for the composition utilizes RT-PCR, in situ hybridization, Northern blotting, or a combination thereof.

12. The method of claim 9, wherein assaying for the polypeptide utilizes antibodies directed to the polypeptide, uses mass spectrometry, or a combination thereof.

13. The method of claim 9, wherein the ovarian cancer is high-grade serous ovarian carcinoma.

14. The method of claim 9, wherein when the sample comprises the CDKN2D-WDFY2 chimeric RNA or an isolated polypeptide produced therefrom and/or comprises the MUC1-KRTCAP2 chimeric RNA or an isolated polypeptide therefrom and/or comprises the BCAM-AKT2 chimeric RNA or an isolated polypeptide therefrom, the individual has high-grade serous ovarian carcinoma or is at risk for having high-grade serous ovarian carcinoma.

15. The method of claim 9, wherein when the sample comprises the CDKN2D-WDFY2 chimeric RNA or an isolated polypeptide produced therefrom and/or comprises the MUC1-KRTCAP2 chimeric RNA or an isolated polypeptide therefrom and/or comprises the BCAM-AKT2 chimeric RNA or an isolated polypeptide therefrom, the individual is provided a suitable treatment for high-grade serous ovarian carcinoma.

16. The method of claim 15, wherein the suitable treatment comprises a therapy that targets the CDKN2D-WDFY2 chimeric RNA and/or the MUC1-KRTCAP2 chimeric RNA and/or the BCAM-AKT2 chimeric RNA.

17. The method of claim 16, wherein the therapy that targets the CDKN2D-WDFY2 chimeric RNA targets the fusion junction of the CDKN2D-WDFY2 chimeric RNA.

18. The method of claim 16, wherein the therapy that targets the MUC1-KRTCAP2 chimeric RNA targets the fusion junction of the MUC1-KRTCAP2 chimeric RNA.

19. The method of claim 16, wherein the therapy that targets the BCAM-AKT2 chimeric RNA targets the fusion junction of the BCAM-AKT2 chimeric RNA.

20. The method of claim 15, wherein the suitable treatment comprises a therapy that targets the polypeptide produced from the CDKN2D-WDFY2 chimeric RNA and/or the MUC1-KRTCAP2 chimeric RNA and/or the BCAM-AKT2 chimeric RNA.

21. The method of claim 10, wherein when the sample comprises the CDKN2D-WDFY2 chimeric RNA or an isolated polypeptide produced therefrom and/or comprises the MUC1-KRTCAP2 chimeric RNA or an isolated polypeptide therefrom and/or comprises the BCAM-AKT2 chimeric RNA or an isolated polypeptide therefrom, a particular therapy for high-grade serous ovarian carcinoma in the individual will be effective.

22. The method of claim 20, wherein the therapy targets the truncated WDFY2 protein from the CDKN2D-WDFY2 chimeric RNA or the signal pathway that is affected by the truncated WDFY2 protein.

23. The method of claim 22, wherein the pathway is the Akt pathway.

24. The method of claim 9, wherein when the sample comprises the CDKN2D-WDFY2 chimeric RNA or an isolated polypeptide produced therefrom and/or comprises the MUC1-KRTCAP2 chimeric RNA or an isolated polypeptide therefrom and/or comprises the BCAM-AKT2 chimeric RNA or an isolated polypeptide therefrom, a particular therapy for high-grade serous ovarian carcinoma in the individual will not be effective.

25. The method of claim 24, wherein the therapy targets the truncated WDFY2 protein from the CDKN2D-WDFY2 chimeric RNA or the signal pathway that is affected by the truncated WDFY2 protein.

26. The method of claim 9, wherein the assaying step comprises polymerase chain reaction.

27. The method of claim 26, wherein the polymerase chain reaction amplifies all or part of the chimeric RNA.

28. The method of claim 26, wherein the polymerase chain reaction amplifies the junction of the chimeric RNA.

29. The method of claim 9, wherein the sample comprises serum, urine, blood, biopsy of cancerous tissue, ascites, pap smear, and a combination thereof.

30. The method of claim 9, wherein the assaying step comprises detection of the polypeptide produced from the CDKN2D-WDFY2 chimeric RNA and/or the polypeptide produced from the MUC1-KRTCAP2 chimeric RNA and/or the polypeptide produced from the BCAM-AKT2 chimeric RNA.

31. The method of claim 30, wherein the method comprises detection of the polypeptide by antibody.

32. The method of claim 9, further comprising the step of performing an additional cancer detection step on a sample from the individual.

33. The method of claim 9, further comprising the step of obtaining the sample from the individual.

34. A method of detecting a particular nucleic acid or polypeptide from a sample from an individual, comprising the step of assaying a sample from the individual for the presence of the composition of claim 1, or assaying a sample from the individual for the presence of a polypeptide produced from the composition of claim 1.

35. The method of claim 34, further comprising the step of obtaining the sample from the individual.

36. The method of claim 34, further comprising the step of identifying a diagnosis, prognosis, and/or treatment for the individual based on the outcome of the assaying step.

37. The method of claim 35, wherein when a chimeric RNA or polypeptide produced therefrom is detected in the sample, the individual has cancer or is at risk for having cancer or is in need of a certain treatment for cancer.

38. The method of claim 36, wherein the cancer is ovarian cancer.

39. The method of claim 36, wherein the cancer is high-grade serous carcinoma.
